SEQUENCE LISTING (20 sequences)

SEQ ID NO: 1 - protein, 15 aa, synthetic construct
RASKSVSTSGFSHMH

SEQ ID NO: 2 - protein, 7 aa, synthetic construct
LASNLES

SEQ ID NO: 3 - protein, 7 aa, synthetic construct
LGSNLES

SEQ ID NO: 4 - protein, 7 aa, synthetic construct
GASNLES

SEQ ID NO: 5 - protein, 9 aa, synthetic construct
QHSRELPRT

SEQ ID NO: 6 - protein, 5 aa, synthetic construct
DYNMD

SEQ ID NO: 7 - protein, 5 aa, synthetic construct
DYNMN

SEQ ID NO: 8 - protein, 5 aa, synthetic construct
DYNMH

SEQ ID NO: 9 - protein, 17 aa, synthetic construct
DINPNTGGTIYNQKFKG

SEQ ID NO: 10 - protein, 9 aa, synthetic construct
REDWYYFDF

SEQ ID NO: 11 - protein, 111 aa, synthetic construct
DIVLTQSPATLALSPGERATLSCRASKSVSTSGFSHMHWYQQKPGQPPKLLIYLASNLESGVPSRFSFSGSGTDFTLNIHPVQEEDAATYYCQHSRELPRTFGGGTKVEIK

SEQ ID NO: 12 - protein, 111 aa, synthetic construct
DIVLTQSPATLSLSPGERATLSCRASKSVSTSGFSHMHWYQQKPGQPPKLLIYLASNLESGVPSRFSFSGSGTDFTLTISSLQPEDVATYYCQHSRELPRTFGGGTKVEIK

SEQ ID NO: 13 - protein, 111 aa, synthetic construct
DIVLTQSPATLSLSPGERATLSCRASKSVSTSGFSHMHWYQQKPGQPPKLLIYLASNLESGVPSRFSGSGSGTDFTLTISSLQPEDVATYYCQHSRELPRTFGGGTKVEIK

SEQ ID NO: 14 - protein, 111 aa, synthetic construct
DIVLTQSPATLSLSPGERATLSCRASKSVSTSGFSHMHWYQQKPGQPPKLLIYLGSNLESGVPSRFSFSGSGTDFTLTISSLQPEDVATYYCQHSRELPRTFGGGTKVEIK

SEQ ID NO: 15 - protein, 111 aa, synthetic construct
DIVLTQSPATLSLSPGERATLSCRASKSVSTSGFSHMHWYQQKPGQPPKLLIYGASNLESGVPSRFSFSGSGTDFTLTISSLQPEDVATYYCQHSRELPRTFGGGTKVEIK

SEQ ID NO: 16 - protein, 118 aa, synthetic construct
EVQLVQSGPELKKPGASVKVPCKASGYTFTDYNMDWVRQPHGKGLEWIGDINPNTGGTIYNQKFKGRATITVDKSTSTAYMELSSLRSEDTSVYYCARREDWYYFDFWGQGTTVTVSS

SEQ ID NO: 17 - protein, 118 aa, synthetic construct
EVQLVQSGAEVKKPGASVKVSCKASGYTFTDYNMDWVRQPPGKGLEWIGDINPNTGGTIYNQKFKGRATITVDKSTSTAYMELSSLRSEDTSVYYCARREDWYYFDFWGQGTTVTVSS

SEQ ID NO: 18 - protein, 118 aa, synthetic construct
EVQLVQSGAEVKKPGASVKVSCKASGYTFTDYNMDWVRQPPGKGLEWIGDINPNTGGTIYNQKFKGRATITVDKSTSTAYMELSSLRSEDTAVYYCARREDWYYFDFWGQGTTVTVSS

SEQ ID NO: 19 - protein, 118 aa, synthetic construct
EVQLVQSGAEVKKPGASVKVSCKASGYTFTDYNMNWVRQPPGKGLEWIGDINPNTGGTIYNQKFKGRATITVDKSTSTAYMELSSLRSEDTAVYYCARREDWYYFDFWGQGTTVTVSS

SEQ ID NO: 20 - protein, 118 aa, synthetic construct
EVQLVQSGAEVKKPGASVKVSCKASGYTFTDYNMHWVRQPPGKGLEWIGDINPNTGGTIYNQKFKGRATITVDKSTSTAYMELSSLRSEDTAVYYCARREDWYYFDFWGQGTTVTVSS

(19) United States
(12) Patent Application Publication
(10) Pub. No.: US 2025/0179190 A1
(43) Pub. Date: Jun. 5, 2025

(54) Integrin Alpha10 Antibody

(71) Applicant: Targinta AB, Lund (SE)

(72) Inventors: Evy Lundgren Åkerlund, Bjärred (SE); Katarzyna Chmielarska Masoumi, Lund (SE); Xiaoli Huang, Hjärup (SE)

(73) Assignee: Targinta AB, Lund (SE)

(21) Appl. No.: 18/842,845

(22) PCT Filed: Mar. 3, 2023
(86) PCT No.: PCT/EP23/55423

§ 371 Date: Aug. 30, 2024

(30) Foreign Application Priority Data
Mar. 3, 2022 (EP) 22159909.5

(51) Int. Cl. C07K 16/28; A61K 9/00; A61K 39/00; A61K 47/68; A61P 35/04; G01N 33/574

(52) U.S. Cl. CPC C07K 16/2842; A61K 9/0019; A61K 47/6809; A61K 47/6821; A61K 47/6849; A61P 35/04; G01N 33/57492; A61K 2039/505; A61K 2039/54; C07K 2317/24; C07K 2317/52; C07K 2317/565; C07K 2317/567; C07K 2317/73; C07K 2317/732; C07K 2317/76; C07K 2317/94; G01N 2333/7055

(57) ABSTRACT

The present invention relates to a novel integrin alphalO antibody, as well as uses thereof in medicine. 25 humanized antibodies (abs) derived from the known mouse mAb365 (the hybridoma deposited under the accession number DSM ACC2583) are disclosed. The ones which bind specifically the integrin alpha 10 beta 1 were selected. Out of the 25 humanized variants 5 were selected as lead candidates (designated TAR-Ab8, TAR-Ab9, TAR-Abl3, TAR-Abl4 and TAR-Ab23) since these 5 entire antibodies were shown to have improved thermal stability as compared to the chimeric antibody designated TAR-Ab0. There is further functional characterization only of TAR-Ab23 which has higher binding affinity than mAb365.

### TECHNICAL FIELD

The present invention relates to a novel integrin alpha10 antibody.

### BACKGROUND

Integrins constitute a major family of cell adhesion receptors and are essential for a wide range of physiological processes, including formation and maintenance of tissue structure, cell migration, proliferation, and differentiation. Integrins also play a prominent role in tumour growth and metastasis. Integrins consists of one alpha and one beta subunit and are assembled from 18 different alpha and 8 different beta subunits which form 24 functionally distinct integrin heterodimers in mammals. Integrins have a large extracellular region consisting of a ligand binding globular head, constructed from both the alpha and beta subunits, and two legs, which are connected to single transmembrane helices and short cytoplasmic domains (Hohenester 2014). Some alpha subunits, including integrin alpha10, have an inserted I-domain (collagen-binding region) in the globular head.

### Integrin Alpha10beta1

Integrin alpha10 (gene name ITGA10) belongs to the collagen-binding integrin subfamily which consists of alpha1, alpha2, alpha10, and alpha11 (Gullberg and Lundgren-Åkerlund 2002). Sequence analysis shows that alpha10 has the highest identity with alpha11 (43%) and an identity of 33% with alpha1 and 31% with alpha2. Integrin alpha10beta1 is mainly expressed on chondrocytes in articular cartilage, in the vertebral column, in trachea and in the cartilage supporting the bronchi (Camper et al 2001) and on some cells in specialised fibrous tissues such as the endosteum (cell lining between bone marrow and bone) and periosteum (cell lining outside the bone), fascia of skeletal muscle and tendon and in the ossification groove of Ranvier likely representing mesenchymal stem cells known to be present in these tissues (Camper et al 2001; Gullberg and Lundgren-Åkerlund 2002; Varas L et al 2007; Lundgren-Åkerlund and Aszòdi 2014). Integrin alpha10beta1 has also been found on isolated mesenchymal stem cells (Varas L et al 2007; Uvebrant et al 2019). The interaction between integrin alpha10beta1 and collagen mediate specific signalling between the cells and the surrounding extracellular matrix similar to the other integrin-extracellular matrix protein interactions (Frith et al 2012, Campbell et al 2011, Heino 2000, Bondreau and Jones 1999, Hering 1999). Knockout studies in mice has demonstrated that integrin α10β1 is important for growth plate formation and growth of long bones during skeletal development (Bengtsson T et al 2005). More recently, the analysis of inbred dog strains, exhibiting a truncating mutation in ITGA10, has demonstrated full-blown canine chondrodysplasia (Kyöstilä et al. 2013).

### Integrin Alpha10beta1 Antibodies for Cancer Therapy

Interactions between integrin receptors on tumor cells and extracellular matrix molecules have been shown to play a key role in tumor cell functions such as cell migration, invasion, proliferation, and survival (Kechagia, J. Z et al., 2019; Moreno-Layseca, P et al., 2019; Malric, L et al., 2017). Although there is still limited information on the role of integrin α10β1 in cancer, recent studies show that it is important for tumor growth and metastasis (Thoren et al. 2019; Masoumi et al., 2021; Ellert-Miklaszewska et al., 2020). Moreover, integrin α10β1 represents a promising target for directed antibody-based cancer therapy due to high cell surface expression in certain cancers such as TNBC and glioblastoma, combined with very low expression in most normal tissues. In accord, function-blocking integrin alpha10beta1-antibodies have been shown to reduce migration, growth and proliferation of cancer cells in preclinical cancer models in tumours of the CNS (WO 2016/133449), breast cancer, in particular triple-negative breast cancer, prostate cancer, pancreas cancer and lung cancer (WO 2020/212416). While the validation of the cancer target has been done with mouse antibodies targeting human integrin α10β1, these are too immunogenic for clinical use due to the generation of human anti-mouse antibodies. The immunogenicity can however be reduced through humanization of the mouse monoclonal antibody. Additionally, a human constant region allows for Fc-mediated effector functions and for a longer serum half-life. The binding affinities of humanized antibodies are however often decreased compared to the original mouse antibody (Hideaki Sanada et al, 2018; Scientific Report; Riechmann L, 1988; Nature; Jones P T et al 1986; Nature; Foote J et al, 1992, J. Mol. Biol).

The objective of the present investigation was to generate a humanized version of the function blocking mouse antibody, mAb365, with potential for future use as a therapeutic agent for the treatment of cancer.

### SUMMARY

The inventors of the present invention have developed a novel humanized integrin alpha10 antibody with unexpected and advantageous features compared to the mouse mAb365. Said antibody is a humanized monoclonal antibody specifically binding to the extracellular I-domain of the integrin alpha10 subunit of integrin alpha10beta1 with the ability to inhibit adhesion, proliferation and migration of cancer cells as well as to reduce tumor growth and metastasis.

Therefore, in one aspect, the present invention relates to an antibody or antigen-binding fragment thereof with binding specificity for integrin alpha10, wherein the antibody or antigen-binding fragment comprises:
a light chain variable region comprising
   a) a CDR-L1 comprising or consisting of an amino acid sequence of SEQ ID NO: 1;
   b) a CDR-L2 comprising or consisting of an amino acid sequence selected from the group consisting of SEQ ID NO: 2, and SEQ ID NO: 4; and
   c) a CDR-L3 comprising or consisting of an amino acid sequence of SEQ ID NO: 5;
and/or
a heavy chain variable region comprising
   a) a CDR-H1 comprising or consisting of an amino acid sequence selected from the group consisting of SEQ ID NO: 7, SEQ ID NO: 6 and SEQ ID NO: 8;
   b) a CDR-H2 comprising or consisting of an amino acid sequence of SEQ ID NO: 9; and
   c) a CDR-H3 comprising or consisting of an amino acid sequence of SEQ ID NO: 10.

In another aspect, the present invention relates to a polynucleotide encoding an antibody or antigen-binding fragment thereof as defined herein or a component polypeptide chain thereof.

In yet another aspect, the present invention relates to a vector comprising a polynucleotide as defined herein.

In yet another aspect, the present invention relates to a recombinant host cell comprising a polynucleotide as defined herein or a vector as defined herein.

In yet another aspect, the present invention relates to a pharmaceutical composition comprising the antibody or antigen-binding fragment thereof as defined herein, the polynucleotide as defined herein, the vector as defined herein, and/or the recombinant host cell as defined herein, in a pharmaceutical composition, wherein the composition further comprises a pharmaceutically-acceptable buffer, diluent, carrier or excipient.

In yet another aspect, the present invention relates to a method for producing an antibody or antigen-binding fragment thereof as defined herein, the method comprising culturing a host cell as defined herein comprising the polynucleotide as defined herein or the vector as defined herein, under conditions which permit expression of the encoded antibody or antigen-binding fragment thereof.

In yet another aspect, the present invention relates to an antibody or antigen-binding fragment thereof as defined herein, a polynucleotide as defined herein, a vector as defined herein, a recombinant host cell as defined herein, and/or a composition as defined herein, for use in medicine.

In yet another aspect, the present invention relates to an antibody or antigen-binding fragment thereof as defined herein, a polynucleotide as defined herein, a vector as defined herein, a recombinant host cell as defined herein, and/or a composition as defined herein, for use in the prevention, treatment, alleviation, detection and/or diagnosis of a disease or disorder susceptible to treatment with an inhibitor of integrin alpha10beta1, and/or wherein the disease or disorder is associated with cells expressing integrin alpha10beta1.

In yet another aspect, the present invention relates to an antibody or antigen-binding fragment thereof as defined herein, a polynucleotide as defined herein, a vector as defined herein, a recombinant host cell as defined herein, and/or a composition as defined herein, for use in inhibiting cell migration, cell proliferation, cell growth, cell survival and/or cell adhesion, wherein targeted cells may be cancer cells and/or cancer-associated cells, which express integrin alpha10beta1.

In yet another aspect, the present invention relates to an in vitro method for the detection of cells expressing integrin alpha10beta1 in a subject, the method comprising:
(a) providing a sample of cells from a subject to be tested, such as biopsy tissue or blood sample;
(b) optionally, extracting and/or purifying the cells present in the sample;
(c) contacting an antibody or antigen-binding fragment thereof as defined herein with cells present in the sample;
(d) determining whether the antibody or antigen-binding fragment thereof binds to the cells
   wherein the binding of the antibody or antigen-binding fragment thereof to the cells is indicative of the presence of a disease or disorder associated with cells expressing integrin alpha10beta1 in the tissue of a subject.

In yet another aspect, the present invention relates to an in vitro method for identifying a patient with a disease or disorder associated with cells expressing integrin alpha10beta1 who would benefit from treatment with an antibody or antigen-binding fragment thereof as defined herein, the method comprising:
a) providing a sample, such as biopsy tissue or blood sample, from a patient to be tested;
b) optionally, extracting and/or purifying the cells present in the sample;
c) contacting an antibody or antigen-binding fragment thereof as defined herein with the sample;
d) determining whether the antibody or antigen-binding fragment thereof binds to an integrin alpha10 domain or fragment thereof,
   wherein the binding of the antibody or antigen-binding fragment thereof to an integrin alpha10 domain or fragment thereof is indicative of a patient who would benefit from treatment with an antibody or antigen-binding fragment thereof as defined herein.

In yet another aspect, the present invention relates to a method for treating a patient with a disease or disorder associated with cells expressing integrin alpha10beta1, the method comprising:
a) selecting a patient identified as having a disease or disorder associated with cells expressing integrin alpha10beta1 as defined herein;
b) administering to said patient a therapeutic agent effective in the treatment of said disease or disorder.

In yet another aspect, the present invention relates to a method for the detection of cells expressing integrin alpha10beta1, the method comprising:
a) contacting an antibody or antigen-binding fragment thereof as defined herein with cells to be analysed for their expression of integrin alpha10beta1;
b) determining whether the antibody or antigen-binding fragment thereof binds to the cells
   wherein the binding of the antibody or antigen-binding fragment thereof to the cells is indicative of the presence of a disease or disorder associated with cells expression integrin alpha10beta1 in the tissue of a subject.

In yet another aspect, the present invention relates to a method for in vivo imaging of the expression of integrin alpha10beta1 in a mammal, the method comprising the steps of
a) Providing a mammal,
b) Providing an antibody or antigen-binding fragment thereof as defined herein,
c) administering the antibody or antigen-binding fragment thereof as defined herein to the mammal so as to allow the antibody or a fragment thereof to bind to an extracellular domain of integrin alpha10beta1 of cells in said mammal,
d) optionally adding a second labelled antibody or a fragment thereof to the sample, wherein the second antibody or a fragment thereof binds to the antibody or a fragment thereof in c),
e) detecting the antibody or antigen-binding fragment thereof as defined herein of said cells in c), or optionally detecting the second labelled antibody or a fragment thereof in d) bound to the antibody or a fragment thereof, and
f) creating an image of the detected antibody or a fragment thereof, thereby imaging the expression of integrin alpha10beta1 on cells in a mammal in vivo.

### DESCRIPTION OF DRAWINGS

FIG. 1: Humanized integrin alpha10 antibody variants inhibit proliferation of C2C12alpha10 cells. C2C12alpha10 cells were seeded in 96-well plates coated with collagen type I and treated with five humanized integrin alpha10 antibody variants and the human isotype control antibody Th301 (5 μg/ml each). Cell proliferation, analyzed by BrdU incorporation, is shown relative to the proliferation of non-treated cells (NT), which was set to 100%.

FIG. 2: Humanized integrin alpha10 antibody variants inhibit adhesion of C2C12alpha10 cells. Cells were pre-incubated with five humanized integrin alpha10 antibody variants and the human isotype control Th301 (5 μg/ml each). The cells were allowed to adhere to plates coated with collage type I. The bar chart shows the data as the mean of three independent cell adhesion experiments ±SE. Cell adhesion is presented relative to the adhesion of non-treated cells (NT), which was set to 100%.

FIG. 3: Humanized integrin alpha10 antibody variants inhibit migration of C2C12alpha10 cells. Spheroids composed of C2C12alpha10 cells were embedded in collagen I gels and treated with five humanized integrin alpha10 antibody variants and the human isotype control Th301 (10 μg/ml each). Migration of cells from the spheroids was analyzed after 24 hours. Relative migration was quantified as migration area difference. Data are expressed as mean±SE. Cell migration is presented relative to the migration of non-treated cells (NT), which was set to 100%.

FIG. 4: Higher cell binding affinity of TAR-Ab23 compared to mAb365. The figure shows binding of the humanised integrin alpha10 antibody TAR-Ab23 and the mouse integrin alpha10 antibody mAb365 to C2C12alpha10 cells (A) triple negative breast cancer cells BT549 (B) and patient derived glioblastoma U3046MG cells (C) at different concentrations. A comparison of the cell binding capacity of the two antibodies at a low antibody concentration (0.1 nM) is presented in figure D. Cells were incubated with antibodies at the indicated concentrations (nM) followed by incubation with secondary antibodies. Immunolabeled cells were analyzed by flow cytometry.

FIG. 5: Higher binding capacity of TAR-Ab23 compared to mAb365 in a competition assay. The figure shows the percentage of mean fluorescence intensity (MFI) in a flow cytometry competition assay, analysing the cell binding capacity of the humanized integrin alpha10 antibody TAR-Ab23 and mouse integrin alpha10 antibody mAb365 to C2C12alpha10 cells and BT549 cancer cells. The antibodies were added simultaneously at a final concentration of 100 nM. The MFI from single antibody staining was set as 100%.

FIG. 6: TAR-Ab23 has improved blocking effect on adhesion, proliferation and migration of triple negative breast cancer compared to mAb365. (A) Adhesion: BT549 cells were treated with 5 μg/ml of the human integrin alpha10 antibody TAR-Ab23 or the mouse integrin alpha10 antibody mAb365 (5 μg/ml each) and then allowed to adhere to collagen IV. Cell adhesion is shown relative to the adhesion of non-treated (NT) cells, which was set to 100%. Data represent averages of triplicate measurements +/−SD. (B) Proliferation: BT549 cancer cells were mixed with 5 μg/ml of TAR-Ab23, mAb365) or the control antibodies human Th301 and mouse mIgG2a and seeded on collagen IV coated dishes. After 48 h cell proliferation was analyzed by BrdU incorporation. The data is shown relative to the proliferation of the respective isotype control antibody, which was set to 100%. (C) Migration: Spheroids composed of BT549 cells were embedded in collagen I gel and treated with 10 μg/mL of TARG10, mAb365 or the control antibodies Th301 and mIgG2a. Migration of the cells was analyzed after 24 h and quantified as migration area difference. Data are expressed as mean±SE of three independent experiments.

FIG. 7: TAR-Ab23 inhibits metastasis of TNBC cells. The TNBC cells, MDA-MB-231 were labelled with Luc/GFP positive cells, and then injected intravenously (2.0×10⁶ cells) into NUDE-NMRI mice. The mice were then treated with 5 mg/kg of TAR-Ab23 or with PBS as a control. Treatment (twice a week) started 3 days after tumor cell injection and continued for 10 weeks. The graph shows a summary of distant metastasis at the termination day to organs including lung and liver. The metastasis was measured using bioluminescence imaging analysis (total flux (P/S). Results are from 5 mice per group.

FIG. 8: In vitro ADCC mediated by TAR-Ab23. (A) The induction of ADCC using Tar-Ab23 on A204 as a target cell with Jurkat-Lucia/NFAT-CD16 effector cell line. (B) The induction of ADCC using Tar-Ab23 low fucose on A204 as a target cell with Jurkat-Lucia/NFAT-CD16 effector cell line. Effect/target cell ratio was 2:1 in all cases. The data is presented as a relative light unit (RLU).

### DEFINITIONS

As used herein, the singular forms “a”, “an” and “the” include plural referents unless the context clearly states otherwise. Thus, for example, reference to “an antibody” includes a plurality of such antibodies. Similarly, “an anti-integrin alpha10 antibody” can also refer to “Anti-integrin alpha10 antibodies”, as for example the antibody variants described in Examples 1 to 9.

As used herein, the term “some embodiments” can include one, or more than one embodiment.

“Integrin alpha10” or “Integrin alpha10 subunit” or “Integrin alpha10 polypeptide” as used herein refers to the alpha10 subunit of the heterodimeric protein integrin alpha10 beta1. This denotation does not exclude the presence of the beta1 subunit bound to the alpha10 subunit thus forming the integrin alpha10 beta1 heterodimer. “Alpha” and “α”, as well as “alpha10” and “alpha 10” are equivalent terms. “Integrin alpha10” as used herein may also refer to the polynucleotide transcript encoding the alpha10 subunit of the heterodimeric protein integrin alpha10beta1, and fragments thereof.

“Anti-integrin alpha10 antibody” or “Integrin alpha10 antibody” or “Anti-integrin alpha10 subunit antibody” as used herein refers to an antibody capable of recognizing and binding to at least the alpha10 integrin of the heterodimeric protein integrin alpha10 beta1. These antibodies may be antibodies that recognize an epitope of the heterodimeric protein integrin alpha10 beta1, wherein the epitope comprises amino acid residues of both the alpha10 and the beta1 integrin polypeptides.

As used herein, “the antibody or antigen-binding fragment of the invention” can be referred to as “the polypeptide of the invention” or “the antibody polypeptide, or antigen-binding fragment thereof”, since an antibody and fragments thereof are polypeptides.

As used herein, the term “an antibody or an antigen-binding fragment thereof”, includes substantially intact antibodies as well as fragments and derivatives of antibodies. An intact antibody can be regarded as an antibody comprising variable light regions, variable heavy regions, constant light regions and constant heavy regions. It further includes chimeric antibodies, humanized antibodies, isolated human antibodies, single chain antibodies, bispecific antibodies, antibody heavy chains, antibody light chains, homodimers and heterodimers of antibody heavy and/or light chains, and antigen-binding fragments and derivatives of the same. Suitable antigen-binding fragments and derivatives include, but are not necessarily limited to, Fv fragments (e.g. single chain Fv and disulphide-bonded Fv), Fab-like fragments (e.g. Fab fragments, Fab′ fragments and F(ab)2 fragments), single variable domains (e.g. VH and VL domains) and domain antibodies (dAbs, including single and dual formats [i.e. dAb-linker-dAb]).

The potential advantages of using antibody fragments, rather than whole antibodies, are several-fold. The smaller size of the fragments may lead to improved pharmacological properties, such as better penetration of solid tissue. Moreover, antigen-binding fragments such as Fab, Fv, ScFv and dAb antibody fragments can be expressed in and secreted from E. coli, thus allowing the facile production of large amounts of the said fragments.

As used herein, the term the “antibody” or “antigen-binding fragment of the invention” is also intended to encompass antibody mimics (for example, non-antibody scaffold structures that have a high degree of stability yet allow variability to be introduced at certain positions). Those skilled in the art of biochemistry will be familiar with many such molecules, as discussed in Gebauer & Skerra, 2009, Curr Opin Chem Biol 13(3): 245-255 (the disclosures of which are incorporated herein by reference). Exemplary antibody mimics include: affibodies (also called Trinectins; Nygren, 2008, FEBS J, 275, 2668-2676); CTLDs (also called Tetranectins; Innovations Pharmac. Technol. (2006), 27-30); adnectins (also called monobodies; Meth. Mol. Biol., 352 (2007), 95-109); anticalins (Drug Discovery Today (2005), 10, 23-33); DARPins (ankyrins; Nat. Biotechnol. (2004), 22, 575-582); avimers (Nat. Biotechnol. (2005), 23, 1556-1561); microbodies (FEBS J, (2007), 274, 86-95); peptide aptamers (Expert. Opin. Biol. Ther. (2005), 5, 783-797); Kunitz domains (J. Pharmacol. Exp. Ther. (2006) 318, 803-809); affilins (Trends. Biotechnol. (2005), 23, 514-522); affimers (Avacta Life Sciences, Wetherby, UK).

As used herein, the term “antigen-binding fragment” refers to fragments of antibodies retaining the ability to specifically bind to an antigen. Examples of antibody fragment of the present invention includes antibody fragments selected from the group consisting of a Fab-fragment, a Fab′ fragment, a F(ab′)2 fragment and an Fv fragment, such as a single-chain variable fragment (scFv) and a single-domain antibody.

The term “amino acid” as used herein includes the standard twenty genetically-encoded amino acids and their corresponding stereoisomers in the ‘D’ form (as compared to the natural ‘L’ form), omega-amino acids and other naturally-occurring amino acids, unconventional amino acids (e.g. α,α-disubstituted amino acids, N-alkyl amino acids, etc.) and chemically derivatised amino acids as described herein. When an amino acid is being specifically enumerated, such as “alanine” or “Ala” or “A”, the term refers to both L-alanine and D-alanine unless explicitly stated otherwise. Other unconventional amino acids may also be suitable components for polypeptides (the antibody or antigen-binding fragment thereof) of the present invention, as long as the desired functional property is retained by the antibody or antigen-binding fragment. For the amino acid sequences shown, each encoded amino acid residue, where appropriate, is represented by a single letter designation, corresponding to the trivial name of the conventional amino acid.

As used herein “expression vector” or “vector” refers to a DNA construct containing a DNA sequence that is operably linked to a suitable control sequence capable of effecting the expression of the DNA in a suitable host. Such control sequences may, e.g., include a promoter to effect transcription, an optional operator sequence to control such transcription, a sequence encoding suitable mRNA ribosome binding sites and sequences which control termination of transcription and translation. The vector may, e.g., be a plasmid, a phage or simply a potential genomic insert. Once transformed into a suitable host, the vector may, e.g., replicate and function independently of the host genome, or may, in some instances, integrate into the genome itself. Expression vectors are designed, for example, as described in Li et al. (Construction strategies for developing expression vectors for recombinant monoclonal antibody production in CHO cells, Mol Biol Rep. 2018 December; 45(6):2907-2912.

“Subject” as used herein denotes a mammal, such as a rodent, a feline, a canine, an equine and a primate. Preferably a subject according to the invention is a human.

A “sample” as used herein encompasses any subject and a variety of sample types obtained from any subject. Examples of samples useful in the disclosed methods include but are not limited to a subject, a liquid tissue sample such as blood, or a solid tissue sample such as biopsy material or tissue cultures or cells derived there from and the progeny thereof. For example, biological samples include cells obtained from a tissue sample collected from a subject. Thus, samples encompass clinical samples, cells in culture, cell supernatants, cell lysates, and tissue samples, e.g. tissue samples from breast tissue, lung tissue, prostate tissue, pancreatic tissue, bone tissue, cartilage tissue, fat tissue, muscle tissue and connective tissue.

A “cancer” as used herein refers to any malignant and/or invasive growth or tumor caused by abnormal cell growth. As used herein “cancer” refers to tumors named for the type of cells that form them. A cancer or tumor comprise of tumor cells or cancer cells. A cancer or tumor comprises also the cancer or tumor microenvironment, which can also comprise MSCs, fibroblasts, endothelial cells, pericytes, adipocytes, immune cells, tumor associated macrophages TAMs. Part of a cancer or tumor might be stroma cells, for example connective tissue cells such as fibroblasts. Examples of solid tumors include but are not limited to sarcomas and carcinomas. The term “cancer” includes but is not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of different type from latter one.

“Detection”, “detect”, “detecting” as used herein includes qualitative and/or quantitative detection (measuring levels) with or without reference to a control, and further refers to the identification of the presence, absence or quantity of a given target, specifically the target of an integrin alpha 10 subunit.

“Inhibition”, as used herein, means that the presence of the antibody of the invention inhibits, in whole or in part, the binding of ligands to the receptor and/or the disablement of a signal the receptor would elicit upon ligand binding. This includes for example down-stream signaling having effect on cellular behavior and processes. “Inhibition” and “blocking” are herein used as equivalent terms. This inhibition is to be understood as comparing the situation where the inhibiting factor (in the case of the present invention the antibody or antigen-binding fragment thereof with binding specificity for integrin alpha10beta1) is present compared to the situation where the inhibiting factor is not present. The person skilled in the art will appreciate that the degree of inhibition can be determined by methods well known in the art, depending on the signaling event or pathway or activity to be measured.

As used herein, “inhibition of signaling” is defined as a reduction of said signaling event, or of, for example, the activity, confirmation, or production of a receptor or biological pathway or molecule activity. It is to be noted that, for the phrase, formulated for example “inhibiting signaling of (for example) integrin alpha10beta1” is used interchangeably to the phrase “inhibiting (for example) integrin alpha10beta1 signaling”.

“ADCC activity” or “Antibody-dependent cellular cytotoxicity activity” as used herein, refers to an activity to damage a target cell (e.g., tumor cell) by activating an effector cell via the binding of the Fc region of an antibody to an Fc receptor existing on the surface of an effector cell such as a killer cell, a natural killer cell, an activated macrophage or the like. An activity of antibodies of the present invention includes ADCC activity. ADCC activity measurements and antitumor experiments can be carried out in accordance using any assay known in the art.

“Pharmaceutically acceptable”, as used herein, refers to a non-toxic material that does not decrease the effectiveness of the integrin alpha10beta1-binding activity of the antibody or antigen-binding fragment of the invention. Pharmaceutically acceptable buffers, carriers, diluents or excipients are well-known in the art.

By “treatment” we include both therapeutic and prophylactic or preventive treatment of the patient. The terms ‘preventive’ or ‘prophylactic’ are used to encompass the use of an antibody or antigen-binding fragment thereof, or formulation thereof, as described herein which either prevents or reduces the likelihood of a neoplastic disorder or disorder, which also includes the prevention or reduction of the spread, dissemination, or metastasis of neoplastic cells in a patient or subject. The term ‘prophylactic’ also encompasses the use of an antibody or antigen-binding fragment thereof, or formulation thereof, as described herein to prevent recurrence of a neoplastic disease or disorder in a patient who has previously been treated for any of these diseases or disorders. “Treatment” and “alleviation” are used interchangeably herein.

By “diagnosis” we include the detection of cells which are associated with a neoplastic disease or disorder, either in vivo (i.e. within the body of a patient) or ex vivo (i.e. within a tissue or cell sample removed from the body of a patient).

By “disorder associated with cells expressing integrin alpha10beta1” we include such diseases or disorders wherein the pathological cells which are responsible, directly or indirectly, for the disorder express integrin alpha10beta1 on the cell surface. It will be appreciated that the cells expressing integrin alpha10beta1 may be immune cells, cells of the connective tissue such as fibroblasts or neoplastic cells (cancer cells), e.g. tumor cells, per se. In addition, such cells include pathological stem cells (i.e. cancer stem cells, or CSCs) and progenitor cells which are responsible, directly or indirectly, for the development of a neoplastic disease or disorder in an individual. Examples of CSCs are disclosed in Visvader & Lindeman, 2008, Nat Rev Cancer 8:755-768, the disclosures of which are incorporated herein by reference.

Alternatively, or in addition, the cells expressing integrin alpha10beta1 may be associated indirectly with the neoplastic disease or disorder, for example, they may mediate cellular processes required for the cells to survive.

“Buffer”, as used herein, refers to mean an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO and TES.

“Diluent”, as used herein, refers to an aqueous or non-aqueous solution with the purpose of diluting the antibody or antigen-binding fragment in the pharmaceutical preparation. The diluent may be one or more of saline, water, polyethylene glycol, propylene glycol, ethanol or oils (such as safflower oil, corn oil, peanut oil, cottonseed oil or sesame oil).

“Adjuvants”, as used herein, refers to any substance whose admixture with an administered antigen increases or otherwise modifies the immune response to said antigen. Suitable adjuvants are well known by those of skill in the art.

“Carriers”, as used herein, refers to scaffold structures, e.g. a polypeptide or a polysaccharide, to which an antigen is capable of being associated. A carrier may be present independently of an adjuvant. Suitable carriers are well known by those of skill in the art.

A ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e., a carrier or administration vehicle. Further, it is intended to mean an amount sufficient to reduce and most preferably prevent, a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art. The administration of the pharmaceutically effective dose can be carried out both by single administration in the form of an individual dose unit or else several smaller dose units and also by multiple administrations of subdivided doses at specific intervals. Alternatively, the does may be provided as a continuous infusion over a prolonged period.

### DETAILED DESCRIPTION

### Integrin Alpha10 Antibody

In an embodiment, the invention relates to an antibody or antigen-binding fragment thereof with binding specificity for integrin alpha10beta1, wherein the antibody or antigen-binding fragment comprises:
a light chain variable region comprising
   a) a CDR-L1 comprising or consisting of an amino acid sequence of SEQ ID NO: 1;
   b) a CDR-L2 comprising or consisting of an amino acid sequence selected from the group consisting of SEQ ID NO: 2, and SEQ ID NO: 4; and
   c) a CDR-L3 comprising or consisting of an amino acid sequence of SEQ ID NO: 5;
and/or
a heavy chain variable region comprising
   a) a CDR-H1 comprising or consisting of an amino acid sequence selected from the group consisting of SEQ ID NO: 7, SEQ ID NO: 6 and SEQ ID NO: 8;
   b) a CDR-H2 comprising or consisting of an amino acid sequence of SEQ ID NO: 9; and
   c) a CDR-H3 comprising or consisting of an amino acid sequence of SEQ ID NO: 10.

In some embodiments, the antibody or antigen-binding fragment thereof comprises:
a light chain variable region comprising
   a) a CDR-L1 consisting of an amino acid sequence of SEQ ID NO: 1;
   b) a CDR-L2 consisting of an amino acid sequence selected from the group consisting of SEQ ID NO: 2 and SEQ ID NO: 4; and
   c) a CDR-L3 consisting of an amino acid of SEQ ID NO: 5;
and/or
a heavy chain variable region comprising
   a) a CDR-H1 consisting of an amino acid sequence selected from the group consisting of SEQ ID NO: 7, SEQ ID NO: 6 and SEQ ID NO: 8;
   b) a CDR-H2 consisting of an amino acid sequence of SEQ ID NO: 9; and
   c) a CDR-H3 consisting of an amino acid sequence of SEQ ID NO: 10.

The antibody or antigen-binding fragment of the invention has specificity for the alpha10 subunit in the integrin alpha10beta1. By “specificity” we mean that the antibody or antigen-binding fragment is capable of binding to integrin alpha10beta1 in vivo, i.e., under the physiological conditions in which integrin alpha10beta1 exists within the human body. Preferably, the antibody or antigen-binding fragment does not bind or has only minor binding to any other protein in vivo. Alternatively, it is meant that the antibody or antigen-binding fragment is capable of binding to integrin alpha10beta1 ex vivo or in vitro. Such binding specificity may be determined by methods well known in the art, such as ELISA, immunohistochemistry, immunoprecipitation, Western blots and flow cytometry using transfected cells expressing integrin alpha10beta1. Advantageously, the antibody or antigen-binding fragment is capable of binding selectively to integrin alpha10beta1, i.e., it binds at least 10-fold more strongly to integrin alpha10beta1 than to any other proteins.

In some embodiments, the antibody or antigen-binding fragment thereof with binding specificity for integrin alpha10beta1 is humanized.

In some embodiments, the antibody or antigen-binding fragment thereof with binding specificity for integrin alpha10 is a human antibody.

In some embodiments, the antibody or antigen-binding fragment thereof with binding specificity for integrin alpha10 is de-immunized.

In some embodiments, the antibody or antigen-binding fragment thereof with binding specificity for integrin alpha10 is humanized and de-immunized.

In some embodiments, the antibody or antigen-binding fragment thereof with binding specificity for integrin alpha10 binds to human integrin alpha10beta1.

In some embodiments, the antibody or antigen-binding fragment thereof with binding specificity for integrin alpha10 binds to non-human integrin alpha10beta1, such as cynomolgus monkey.

In some embodiments, the antibody or antigen-binding fragment thereof with binding specificity for integrin alpha10 binds integrin alpha10beta1 expressed on the surface of a cell.

In some embodiments, the antibody or antigen-binding fragment thereof with binding specificity for integrin alpha10 binds to an epitope on the extracellular domain-I of the subunit alpha10 of integrin alpha10beta1.

In some embodiments, the antibody or antigen-binding fragment thereof with binding specificity for integrin alpha10beta1 binds to extracellular domain-I of the subunit alpha10 of integrin alpha10beta1.

In some embodiments, as discussed above, the antibody or antigen-binding fragment of the invention comprises or consists of an antibody mimic selected from the group comprising or consisting of affibodies, tetranectins (CTLDs), adnectins (monobodies), anticalins, DARPins (ankyrins), avimers, iMabs, microbodies, peptide aptamers, Kunitz domains and affilins.

Persons skilled in the art will further appreciate that the invention also encompasses modified versions of antibodies and antigen-binding fragments thereof, whether existing now or in the future, e.g., modified by the covalent attachment of polyethylene glycol or another suitable polymer (see below).

### Integrin Alpha10 Polypeptide

Integrins are heterodimers consisting of an alpha and a beta polypeptide. The integrin alpha10 beta1 heterodimer may be detected by integrin alpha10-specific antibodies and integrin alpha10 binding peptides and proteins.

In some embodiments, the integrin alpha10 polypeptide is a part of an integrin alpha10beta1 heterodimer.

In some embodiments, the integrin alpha10 polypeptide is expressed on the surface of the cells.

The integrin alpha10 beta1 was originally identified as a collagen type II binding receptor on chondrocytes in 1998 (Camper et al., 1998). In vitro studies have demonstrated its binding to other collagen subtypes and to laminin (Lundgren-Åkerlund Book chapter and Thoren et al). Immunohistochemical analysis during development and in adult tissues has demonstrated a restricted localization to cartilage-containing tissues and to some fibrous tissues (Camper et al. 1998, Camper et al., 2001). Knockout mice lacking the marker have disorganized growth plates, decreased collagen in the matrix and shorter long-bones, further supporting its cell structural importance (Bengtsson et al., 2005). The amino acid sequence, variants, isoforms and sequence annotations can be found in Uniprot accession no O75578 (ITA10_HUMAN).

Integrin alpha10beta1 receptors transmit, upon binding to the extracellular ligand, intracellular signals that promote cell adhesion, migration, survival, proliferation, tumor growth and metastasis. Inhibition of the receptor thus impedes adhesion, migration, survival, proliferation, tumor growth and metastasis. This may be important for the treatment of many proliferative diseases such as cancer and inflammatory diseases.

In some embodiments, integrin alpha10 is a naturally occurring variant of integrin alpha10 polypeptide, an isoform of integrin alpha10 polypeptide or a splice variant of an integrin alpha10 polypeptide.

Integrin alpha10 can also be detected on nucleotide level by analyzing a sample for the presence of e.g. mRNA transcripts which upon translation generates an integrin alpha10 antigen as defined herein above.

### CDRs

The antibody of the present invention is defined by its characteristic complementarity-determining region (CDR) sequences. There are several approaches for defining the CDR sequences of an antibody. The CDRs of the antibody of the present invention have been defined using definition according to Kabat.

The person skilled in the art will appreciate that a set of 6 CDRs (CDR-L1, CDR-L2, CDR-L3, CDR-H1, CDR-H2, CDR-H3) may be defined according to Kabat.

Further, the person skilled in the art will appreciate that it is possible to define the CDRs of the antibody of the invention by other approaches, for example by definition of CDRs according to Chothia (Al-Lazikani et al., (1997) JMB 273, 927-948), Martin (Enhanced Chotia), Gelfand or Honneger. Further approaches exist, such as the AbM definition (combination of Kabat definition and Chothia definition used by Oxford Molecular's AbM antibody modelling software) or the contact definition (based on analysis of crystal structures). See, e.g., Kabat et al. (Sequences of Proteins of Immunological Interest, 1987 and 1991, NIH, Bethesda, Md.), Lefranc et al. (IMGT unique numbering for immunoglobulin and T cell receptor constant domains and Ig superfamily C-like domains, Dev Comp Immunol. 2005; 29(3):185-203) and Dondelinger et al. (Understanding the Significance and Implications of Antibody Numbering and Antigen-Binding Surface/Residue Definition, Front. Immunol., 16 Oct. 2018).

The person skilled in the art could, when provided with the Kabat CDRs as presented herein, use known information to list other CDR naming conventions or approaches (like Chothia). Thus, all CDR naming conventions or approaches are encompassed.

In certain cases, it can be beneficial to define the CDRs according to one numbering system, such as Kabat. Often, these CDR sequences are short (shorter than, for example, an approach combining numbering systems), thus providing the core sequences critical for binding. In other cases, it can be beneficial to use a combination of, for example, IMGT and Kabat CDR sequences.

However, the person skilled in the art will appreciate that a low level of mutation (typically, just one or two amino acids) within a CDR sequence may be tolerated without loss of the specificity of the antibody or antigen-binding fragment for integrin alpha10.

In some embodiments, the antibody or antigen-binding fragment thereof with binding specificity for integrin alpha10 comprises a light chain variable region comprising
a) a CDR-L1 comprising or consisting of an amino acid sequence of SEQ ID NO: 1;
b) a CDR-L2 comprising or consisting of an amino acid sequence selected from the group consisting of SEQ ID NO: 2, and
c) a CDR-L3 comprising or consisting of an amino acid sequence of SEQ ID NO: 5; and
a heavy chain variable region comprising
a) a CDR-H1 comprising or consisting of an amino acid sequence selected from the group consisting of SEQ ID NO: 7,
b) a CDR-H2 comprising or consisting of an amino acid sequence of SEQ ID NO: 9; and
c) a CDR-H3 comprising or consisting of an amino acid sequence of SEQ ID NO: 10; or an amino acid sequence having at least 70% sequence identity to SEQ ID NO: 1, SEQ ID NO: 2, SEQ ID NO: 5, SEQ ID NO: 7, or SEQ ID NO: 9, SEQ ID NO: 10, for example at least 80%, 85%, 90%, 95%, 98% or 99% sequence identity.

In some embodiments, the antibody or antigen-binding fragment thereof with binding specificity for integrin alpha10 comprises CDRs as described above (comprising or consisting of the amino acid sequence selected from the group consisting of SEQ ID NOs 1 to 10), wherein any one of the amino acids of the CDRs has been altered for another amino acid, for example, with the proviso that no more than 2 amino acids have been so altered, such as 1 amino acid.

### Light Chain Variable Regions

In some embodiments, the antibody or antigen-binding fragment thereof with binding specificity for integrin alpha10 comprises a light chain variable region comprising or consisting of
a) the amino acid sequence of SEQ ID NO: 13,
or an amino acid sequence having at least 85% sequence identity to SEQ ID NO: 13, for example at least 90%, 95%, 98% or 99% sequence identity; or
b) an amino acid sequence selected from the group consisting of SEQ ID NO: 11, SEQ ID NO:12; SEQ ID NO: 13; SEQ ID NO: 14 and SEQ ID NO:15, or an amino acid sequence having at least 85% sequence identity to SEQ ID NO: 11, SEQ ID NO:12; SEQ ID NO: 13; SEQ ID NO: 14 and SEQ ID NO:15, for example at least 90%, 95%, 98% or 99% sequence identity to any one of SEQ ID NO: 11, SEQ ID NO:12; SEQ ID NO: 14 and SEQ ID NO:15.

In some embodiments, the antibody or antigen-binding fragment thereof with binding specificity for integrin alpha10 comprises a light chain variable region comprising or consisting of the amino acid sequence of SEQ ID NO: 13, or an amino acid sequence having at least 85% sequence identity to SEQ ID NO: 13, for example at least 90%, 95%, 98% or 99% sequence identity.

Percent identity (or sequence identity) can be determined by, for example, the LALIGN program at the Expasy facility site (http://www.ch.embnet.org/software/LALIGN_form.html) using as parameters the global alignment option, scoring matrix BLOSUM62, opening gap penalty −14, extending gap penalty −4. Alternatively, the percent sequence identity between two polypeptides, such as parts of an antibody, may be determined using suitable computer programs, for example the GAP program of the University of Wisconsin Genetic Computing Group and it will be appreciated that percent identity is calculated in relation to polypeptides whose sequence has been aligned optimally.

The alignment may alternatively be carried out using the Clustal W program. The parameters used may be as follows:
Fast pair-wise alignment parameters: K-tuple (word) size; 1, window size; 5, gap penalty; 3, number of top diagonals; 5. Scoring method: x percent.
Multiple alignment parameters: gap open penalty; 10, gap extension penalty; 0.05.
Scoring matrix: BLOSUM.

Alternatively, the BESTFIT program may be used to determine local sequence alignments.

The person skilled in the art will consider further alterations to the above described light chain variable regions, for example to further optimize the antibody or antigen-binding fragment. Usually, the person skilled in the art will, as done during humanization and de-immunization procedures, consider to alter amino acids in the framework regions, i.e., outside the epitope binding CDR regions, thereby usually not altering the CDR regions.

In some embodiments, the antibody or antigen-binding fragment thereof with binding specificity for integrin alpha10 comprises a light chain variable region as described above, wherein any one of the amino acids of the framework region of the light chain variable region has been altered for another amino acid, with the proviso that no more than 5 amino acids have been so altered, such as 4 amino acids, no more than 3 amino acids, such as 2 amino acids or no more than 1 amino acid.

### Heavy Chain Variable Regions

In some embodiments, the antibody or antigen-binding fragment thereof with binding specificity for integrin alpha10 comprises a heavy chain variable region comprising or consisting of
a) the amino acid sequence of SEQ ID NO: 19; or an amino acid sequence having at least 85% sequence identity to SEQ ID NO: 19 for example at least 90%, 95%, 98% or 99% sequence identity; or
b) an amino acid sequence selected from the group consisting of SEQ ID NO: 16, SEQ ID NO: 17, SEQ ID NO: 18, and SEQ ID NO: 20; or an amino acid sequence having at least 85% sequence identity to SEQ ID NO: 16, SEQ ID NO: 17, SEQ ID NO: 18, and SEQ ID NO: 20, for example at least 90%, 95%, 98% or 99% sequence identity to any one of SEQ ID NO: 16, SEQ ID NO: 17, SEQ ID NO: 18, and SEQ ID NO: 20.

In some embodiments, the antibody or antigen-binding fragment thereof with binding specificity for integrin alpha10 comprises a heavy chain variable region comprising or consisting of the amino acid sequence of SEQ ID NO: 19; or an amino acid sequence having at least 85% sequence identity to SEQ ID NO: 19 for example at least 90%, 95%, 98% or 99% sequence identity.

The person skilled in the art will consider further alterations to the above described heavy chain variable regions, for example to further optimize the antibody or antigen-binding fragment. Usually, the person skilled in the art will, as done during humanization and de-immunization procedures, consider to alter amino acids in the framework regions, i.e. outside the epitope binding CDR regions, thereby not altering the CDR regions.

In some embodiments, the antibody or antigen-binding fragment thereof with binding specificity for integrin alpha10beta1 comprises a heavy chain variable region as described above, wherein any one of the amino acids of the framework region of the heavy chain variable region has been altered for another amino acid, with the proviso that no more than 5 amino acids have been so altered, such as 4 amino acids, no more than 3 amino acids, such as 2 amino acids or no more than 1 amino acid.

### Combination of Variable Light and Variable Heavy Chains

The person skilled in the art will appreciate that any of the above described variants of light chain variable regions can be combined with any of the above described variants of heavy chain variable regions.

In some embodiments, the antibody or antigen-binding fragment thereof with binding specificity for integrin alpha10 comprises a light chain variable region which comprises or consists of SEQ ID NO: 13 and a heavy chain variable region which comprises or consists of SEQ ID NO: 19,

or an amino acid sequence having at least 70% sequence identity to SEQ ID NO: 13 or SEQ ID NO: 19, for example at least 80%, 85%, 90%, 95%, 98% or 99% sequence identity.

In some embodiments, the antibody or antigen-binding fragment thereof with binding specificity for integrin alpha10 comprises:
a) a light chain variable region which comprises or consists of the amino acid sequence of SEQ ID NO: 11 and a heavy chain variable region which comprises or consists of the amino acid sequence of SEQ ID NO: 16;
b) a light chain variable region which comprises or consists of the amino acid sequence of SEQ ID NO: 11 and a heavy chain variable region which comprises or consists of the amino acid sequence of SEQ ID NO: 17;
c) a light chain variable region which comprises or consists of the amino acid sequence of SEQ ID NO: 11 and a heavy chain variable region which comprises or consists of the amino acid sequence of SEQ ID NO: 18;
d) a light chain variable region which comprises or consists of the amino acid sequence of SEQ ID NO: 11 and a heavy chain variable region which comprises or consists of the amino acid sequence of SEQ ID NO: 19;
e) a light chain variable region which comprises or consists of the amino acid sequence of SEQ ID NO: 11 and a heavy chain variable region which comprises or consists of the amino acid sequence of SEQ ID NO: 20;
f) a light chain variable region which comprises or consists of the amino acid sequence of SEQ ID NO: 12 and a heavy chain variable region which comprises or consists of the amino acid sequence of SEQ ID NO: 16;
g) a light chain variable region which comprises or consists of the amino acid sequence of SEQ ID NO: 12 and a heavy chain variable region which comprises or consists of the amino acid sequence of SEQ ID NO: 17;
h) a light chain variable region which comprises or consists of the amino acid sequence of SEQ ID NO: 12 and a heavy chain variable region which comprises or consists of the amino acid sequence of SEQ ID NO: 18;
i) a light chain variable region which comprises or consists of the amino acid sequence of SEQ ID NO: 12 and a heavy chain variable region which comprises or consists of the amino acid sequence of SEQ ID NO: 19;
j) a light chain variable region which comprises or consists of the amino acid sequence of SEQ ID NO: 12 and a heavy chain variable region which comprises or consists of the amino acid sequence of SEQ ID NO: 20;
k) a light chain variable region which comprises or consists of the amino acid sequence of SEQ ID NO: 13 and a heavy chain variable region which comprises or consists of the amino acid sequence of SEQ ID NO: 16;
l) a light chain variable region which comprises or consists of the amino acid sequence of SEQ ID NO: 13 and a heavy chain variable region which comprises or consists of the amino acid sequence of SEQ ID NO: 17;
m) a light chain variable region which comprises or consists of the amino acid sequence of SEQ ID NO: 13 and a heavy chain variable region which comprises or consists of the amino acid sequence of SEQ ID NO: 18;
n) a light chain variable region which comprises or consists of the amino acid sequence of SEQ ID NO: 13 and a heavy chain variable region which comprises or consists of the amino acid sequence of SEQ ID NO: 19;
o) a light chain variable region which comprises or consists of the amino acid sequence of SEQ ID NO: 13 and a heavy chain variable region which comprises or consists of the amino acid sequence of SEQ ID NO: 20;
p) a light chain variable region which comprises or consists of the amino acid sequence of SEQ ID NO: 14 and a heavy chain variable region which comprises or consists of the amino acid sequence of SEQ ID NO: 16;
q) a light chain variable region which comprises or consists of the amino acid sequence of SEQ ID NO: 14 and a heavy chain variable region which comprises or consists of the amino acid sequence of SEQ ID NO: 17;
r) a light chain variable region which comprises or consists of the amino acid sequence of SEQ ID NO: 14 and a heavy chain variable region which comprises or consists of the amino acid sequence of SEQ ID NO: 18;
s) a light chain variable region which comprises or consists of the amino acid sequence of SEQ ID NO: 14 and a heavy chain variable region which comprises or consists of the amino acid sequence of SEQ ID NO: 19;
t) a light chain variable region which comprises or consists of the amino acid sequence of SEQ ID NO: 14 and a heavy chain variable region which comprises or consists of the amino acid sequence of SEQ ID NO: 20;
u) a light chain variable region which comprises or consists of the amino acid sequence of SEQ ID NO: 15 and a heavy chain variable region which comprises or consists of the amino acid sequence of SEQ ID NO: 16;
v) a light chain variable region which comprises or consists of the amino acid sequence of SEQ ID NO: 15 and a heavy chain variable region which comprises or consists of the amino acid sequence of SEQ ID NO: 17;
w) a light chain variable region which comprises or consists of the amino acid sequence of SEQ ID NO: 15 and a heavy chain variable region which comprises or consists of the amino acid sequence of SEQ ID NO: 18;
x) a light chain variable region which comprises or consists of the amino acid sequence of SEQ ID NO: 15 and a heavy chain variable region which comprises or consists of the amino acid sequence of SEQ ID NO: 19; or
y) a light chain variable region which comprises or consists of the amino acid sequence of SEQ ID NO: 15 and a heavy chain variable region which comprises or consists of the amino acid sequence of SEQ ID NO: 20;
   or an amino acid sequence having at least 70% sequence identity to any one of SEQ ID NO: 11 to SEQ ID NO: 20, for example at least 80%, 85%, 90%, 95%, 98% or 99% sequence identity.

These combinations of light and heavy chain variable regions are, for example, part of the humanized antibody variants as described in Examples 1 and 2.

In some embodiments, the antibody or antigen-binding fragment thereof with binding specificity for integrin alpha10 comprises a heavy chain variable region as described above, wherein any one of the amino acids of the framework region of the light chain variable region and/or the heavy chain variable region has been altered for another amino acid, with the proviso that no more than 5 amino acids have been so altered, such as 4 amino acids, no more than 3 amino acids, such as 2 amino acids or no more than 1 amino acid.

### Constant Regions and Fc Part

The person skilled in the art will appreciate that any light constant region known in the art can be combined with any of the above described variants of light variable regions, and that any heavy constant region known in the art can be combined with any of the above described variants of heavy variable regions, to form a full antibody.

In some embodiments, the antibody or antigen-binding fragment thereof with binding specificity for integrin alpha10 comprises a light chain constant region, or part thereof.

In some embodiments, the antibody or antigen-binding fragment thereof with binding specificity for integrin alpha10 comprises a light chain constant region which is of a kappa or lambda light chain.

In some embodiments, the antibody or antigen-binding fragment thereof with binding specificity for integrin alpha10 comprises a heavy chain constant region, or part thereof.

In some embodiments, the antibody or antigen-binding fragment thereof with binding specificity for integrin alpha10 comprises a heavy chain constant region which is selected from the group consisting of α, δ, γ, ε and μ.

In some embodiments, the antibody or antigen-binding fragment thereof with binding specificity for integrin alpha10 comprises a heavy chain constant region which is of a human immunoglobulin isotype selected from the group consisting of IgA, IgD, IgG, IgE and IgM.

In some embodiments, the antibody or antigen-binding fragment thereof with binding specificity for integrin alpha10 comprises a heavy chain constant region which is of an IgG human immunoglobulin isotype.

In some embodiments, the antibody or antigen-binding fragment thereof with binding specificity for integrin alpha10 is an IgG antibody.

In some embodiments, the antibody or antigen-binding fragment thereof with binding specificity for integrin alpha10 comprises a heavy chain constant region which is of a human immunoglobulin subclass selected from the group consisting of IgG1, IgG2, IgG3 and IgG4.

In some embodiments the antibody or antigen-binding fragment thereof with binding specificity for integrin alpha10 comprises or consists of a light chain constant region and/or a heavy chain constant region wherein any one of the amino acids of the above mentioned light chain constant region and/or above mentioned heavy chain constant region has been altered for another amino acid, with the proviso that no more than 5 amino acids have been so altered, such as 4 amino acids, no more than 3 amino acids, such as 2 amino acids or no more than 1 amino acid.

In some embodiments, the antibody or antigen-binding fragment of the present invention comprises a CH1, CH2 and/or CH3 region of an IgG heavy chain (such as an IgG1, IgG2, IgG3 or IgG4 heavy chain). Thus, the antibody or antigen-binding fragment may comprise part or all of the constant regions from an IgG1 heavy chain. For example, the antibody or antigen-binding fragment may be a Fab fragment comprising CH1 and CL constant regions, combined with any of the above-defined heavy and light variable regions, respectively.

Likewise, the above-defined antibodies or antigen-binding fragments of the invention may further comprise a light chain constant region, or part thereof. For example, the antibody or antigen-binding fragment may comprise a CL region from a kappa or lambda light chain.

In some embodiments, the antibody or antigen-binding fragment thereof with binding specificity for integrin alpha10beta1 comprises or consists of
any one of the above mentioned light chain variable regions; and/or
any one of the above mentioned heavy chain variable regions; and/or
any one of the above mentioned light chain constant regions; and/or
any one of the above mentioned heavy chain constant region.

In some embodiments, the antibody or antigen-binding fragment thereof with binding specificity for integrin alpha10 comprises an Fc region. Fc region may also be referred to as Fc domain.

In some embodiments, the antibody or antigen-binding fragment thereof with binding specificity for integrin alpha10 comprises an Fc region which is naturally occurring.

In some embodiments, the antibody or antigen-binding fragment thereof with binding specificity for integrin alpha10 comprises an Fc region which is non-naturally occurring.

In some embodiments, the antibody or antigen-binding fragment thereof with binding specificity for integrin alpha10 comprises an Fc region with a modified, for example with a mutated, IgG constant region.

The Fc region may be naturally-occurring (e.g. part of an endogenously produced antibody) or may be artificial (e.g. comprising one or more point mutations relative to a naturally-occurring Fc region).

As is well documented in the art, the Fc region of an antibody mediates its serum half-life and effector functions, such as complement-dependent cytotoxicity (CDC), antibody-dependent cellular cytotoxicity (ADCC) and antibody-dependent cell phagocytosis (ADCP).

Engineering the Fc region of a therapeutic monoclonal antibody or Fc fusion protein allows the generation of molecules that are better suited to the pharmacology activity required of them (Strohl, 2009, Curr Opin Biotechnol 20(6):685-91, the disclosures of which are incorporated herein by reference). Improved Fc-binding can also be obtained by applying certain culturing conditions, for example fucose-low conditions.

### Inhibition of Biological Activities

In some embodiments, the integrin alpha10 antibody of the present invention is an antibody capable of inhibiting the biological and functional activity of integrin alpha10beta1.

In some embodiments, the antibody or antigen-binding fragment thereof with binding specificity for integrin alpha10 is capable of inhibiting signaling of the integrin alpha10beta1. The person skilled in the art will appreciate that inhibition occurs upon binding of the antibody or antigen-binding fragment to the antibody epitope on integrin alpha10. Integrin alpha10beta1 is involved in both outside in and inside out signalling, and both signalling can be inhibited by using an antibody according to the present disclosure.

In some embodiments, the antibody or antigen-binding fragment thereof with binding specificity for integrin alpha10 is capable of inhibiting signaling upon binding to integrin alpha10.

In some embodiments, the antibody or antigen-binding fragment thereof with binding specificity for integrin alpha10 is capable of inhibiting cell survival, cell adhesion, cell proliferation, cell growth, cell migration, or any combination thereof, of cells expressing integrin alpha10beta1. The person skilled in the art will appreciate that inhibition occurs upon binding of the antibody or antigen-binding fragment to the epitope on integrin alpha10.

In some embodiments, the antibody or antigen-binding fragment thereof with binding specificity for integrin alpha10 is capable of inhibiting cell survival of cells expressing integrin alpha10beta1.

In some embodiments, the antibody or antigen-binding fragment thereof with binding specificity for integrin alpha10 is capable of inhibiting cell adhesion of cells expressing integrin alpha10beta1.

In some embodiments, the antibody or antigen-binding fragment thereof with binding specificity for integrin alpha10 is capable of inhibiting cell proliferation of cells expressing integrin alpha10beta1.

In some embodiments, the antibody or antigen-binding fragment thereof with binding specificity for integrin alpha10 is capable of inhibiting cell growth of cells expressing integrin alpha10beta1.

In some embodiments, the antibody or antigen-binding fragment thereof with binding specificity for integrin alpha10beta1 is capable of inhibiting cell migration of cells expressing integrin alpha10beta1.

In some embodiments, the antibody or antigen-binding fragment thereof with binding specificity for integrin alpha10 is capable of inhibiting metastasis of cells expressing integrin alpha10beta1. The person skilled in the art will appreciate that inhibition occurs upon binding of the antibody or antigen-binding fragment to the epitope on integrin alpha10.

In some embodiments, the antibody or antigen-binding fragment thereof with binding specificity for integrin alpha10 is capable of inhibiting tumour growth of tumours comprising cells expressing integrin alpha10 in the tumour itself and/or in the tumour microenvironment.

The person skilled in the art will appreciate that binding of the antibody or antigen binding fragment thereof to integrin alpha10beta1 can affect integrin alpha10beta1 in different ways, for example on a direct molecular level and/or on a conformational level. For example, binding of the antibody or antigen binding fragment thereof to integrin alpha10beta1 may block ligand binding and thus change the integrin alpha10beta1 signaling; it may also result in internalization of the antibody or antigen binding fragment thereof.

The person skilled in the art will appreciate that not all integrin alpha10 binding antibodies, binding to any one epitope of integrin alpha10beta1, can inhibit cell survival, cell adhesion, cell proliferation, cell growth, cell migration, metastasis, tumour growth, integrin alpha10beta1 signaling and/or any combination thereof. It is an achievement of the present invention to provide such an antibody.

The person skilled in the art will appreciate that inhibition of a biological activity, such as cell survival, cell adhesion, cell proliferation, cell growths, cell migration, metastasis, tumour growth, integrin alpha10beta1 signaling and/or any combination thereof, can be of a varying degree. Inhibition can be complete or essentially complete. Inhibition can also be partial, such as decreasing, such as non-complete. “Essentially complete” is to be understood as “complete” given uncertainties of assessing completeness associated with the methodology used to measure the inhibition of a biological activity.

For example, a biological activity, such as cell survival, cell adhesion, cell proliferation, cell growth, cell migration, tumour growth, metastasis, integrin alpha10beta1 signaling and/or any combination thereof, may be inhibited by at least 10%, 20%, 30%, 50%, 60%, 75% 80%, 85%, 90%, 95%, 98%, 99% or more relative to the biological activity in the absence of the antibody or antigen-binding fragment of the invention.

In some embodiments, inhibition of a biological activity, such as cell survival, cell adhesion, cell proliferation, cell growth, cell migration, tumour growth, metastasis, integrin alpha10beta1 signaling and/or any combination thereof, is between 10 to 100% relative to the biological activity in the absence of the antibody or antigen-binding fragment of the invention. More preferably the inhibition of the biological activity is between 25 to 100%. Even more preferably the inhibition of the biological activity is between 50 to 100%.

The degree of inhibition of a biological activity, such as cell survival, cell adhesion, cell proliferation, cell growth, cell migration, tumour growth, metastasis, integrin alpha10beta1 signaling and/or any combination thereof, by the antibody or antigen-binding fragment of the invention may be determined using methods well known in the art, for example by the methods used in Examples 7 and 8.

In preferred embodiments, the antibody or antigen-binding fragment thereof with binding specificity for integrin alpha10 is capable of essentially completely inhibiting metastasis.

The antibody might further inhibit metastasis to a certain degree, meaning not essentially completely, meaning partially.

In some embodiments, the antibody or antigen-binding fragment thereof with binding specificity for integrin alpha10beta1 is capable of partially inhibiting metastasis.

The person skilled in the art will appreciate that an integrin alpha10 antibody, apart from the named properties described above, can exhibit one or more further functions. These functions can be conveyed by the constant region of the antibody or the Fc region. Examples of these functions are antibody-dependent cell-mediated cytotoxicity (ADCC) and/or antibody-dependent cellular phagocytosis (ADCP) and/or complement-dependent cytotoxicity (CDC), thereby leading to the killing of target cells, such as integrin alpha10beta1-expressing tumor cells.

In some embodiments, the antibody or antigen-binding fragment thereof with binding specificity for integrin alpha10 is capable of inducing ADCC of cells expressing integrin alpha10beta1.

In some embodiments, the antibody or antigen-binding fragment thereof with binding specificity for integrin alpha10 is a low fucose variant, and it is capable of inducing ADCC of cells expressing integrin alpha10beta1.

In some embodiments, the antibody or antigen-binding fragment thereof with binding specificity for integrin alpha10beta1 is capable of inducing ADCP of cells expressing integrin alpha10beta1.

In some embodiments, the antibody or antigen-binding fragment thereof with binding specificity for integrin alpha10beta1 is capable of inducing CDC of cells expressing integrin alpha10beta1.

As explained in detail in the description below, antibodies can be subjected to modifications to optimize certain properties.

### Modifications of the Antibody

In some embodiments, the antibody or antigen-binding fragment thereof with binding specificity for integrin alpha10 further comprises a moiety for increasing the in vivo half-life of the agent.

In some embodiments, the moiety for increasing the in vivo half-life of the antibody or antigen-binding fragment thereof with binding specificity for integrin alpha10 is selected from the group consisting of polyethylene glycol (PEG), human serum albumin, glycosylation groups, fatty acids and dextran.

In some embodiments, the antibody or antigen-binding fragment thereof with binding specificity for integrin alpha10 is PEGylated.

In some embodiments, the antibody or antigen-binding fragment thereof with binding specificity for integrin alpha10 fragment is covalently bound, directly or indirectly, to a functional moiety such as a cytotoxic or detectable moiety.

In some embodiments, the antibody or antigen-binding fragment thereof with binding specificity for integrin alpha10 comprises a cytotoxic moiety.

In some embodiments, the cytotoxic moiety comprises or consists of a radioisotope.

In some embodiments, the radioisotope is selected from the group consisting of beta-emitters, auger-emitters, conversion electron-emitters, alpha-emitters, and low photon energy-emitters.

In some embodiments, the radioisotope has an emission pattern of locally absorbed energy that creates a high dose absorbance in the vicinity of the agent.

In some embodiments, the radioisotope is selected from the group consisting of long-range beta-emitters, such as ⁹⁰Y, ³²P, ¹⁸⁶Re/¹⁸⁶Re; ¹⁶⁶Ho, ⁷⁶As/⁷⁷As, ¹⁵³Sm; medium range beta-emitters, such as ¹³¹I, ¹⁷⁷Lu, ⁶⁷Cu, ¹⁶¹Tb; low-energy beta-emitters, such as ⁴⁵Ca, ³⁵S or ¹⁴C; conversion or auger-emitters, such as ⁵¹Cr, ⁶⁷Ga, ⁹⁹Tc^{m}, ¹¹¹In, ¹²³I, ¹²⁵I, ²⁰¹Tl; and alpha-emitters, such as ²¹²Bi, ²¹³Bi, ²²³Ac, and ²²¹At.

In some embodiments, the antibody or antigen-binding fragment thereof with binding specificity for integrin alpha10beta1 comprises a cytotoxic moiety which comprises or consists of a cytotoxic drug.

In some embodiments, the cytotoxic drug is selected from the group consisting of a cytostatic drug; an anti-androgen drug; cortisone and derivatives thereof; a phosphonate; a testosterone-5-α-reductase inhibitor; a boron addend; a cytokine; thapsigargin and its metabolites; a toxin (such as saporin or calicheamicin); a chemotherapeutic agent (such as an antimetabolite); or any other cytotoxic drug useful in the treatment of neoplastic disorders.

In some embodiments, the neoplastic disease or disorder is any of the neoplastic diseases or disorders defined in detail in the section “Clinical conditions” herein. In some embodiments, the cytotoxic moiety is selected from a group consisting of a toxin, a chemotherapeutic agent and a radioactive agent, or combinations thereof.

In some embodiments, the toxin is selected from the group selected from microtubule toxins, DNA toxins and transcription toxins.

In some embodiments, the microtubule toxin which is selected from the group consisting of Auristatin-based toxins, Maytansinoid-based toxins, Tubulysins-based toxins and Eribulin

In some embodiments, the cytotoxic moiety is a DNA toxin selected from the group consisting of DNA minor-groove binding agents, DNA minor-groove binding alkylating agents, DNA alkylating agents and DNA-cleaving agents. For example, in some embodiments the cytotoxic moiety is a DNA toxin selected from the group consisting of Pyrrolobenzodiazepine (PBD), Duocarmycin, Duocarmycin analogues, Indolino-benzodiazepine, Calicheamicins, Irinotecan and Exatecan derivatives.

In some embodiments, the transcription toxin is an RNA polymerase II inhibiting agent.

In some embodiments, the transcription toxin is selected from the group consisting of Doxorubicin, Doxorubicin derivatives and Amanitin.

In some embodiments, the antibody or antigen-binding fragment thereof with binding specificity for integrin alpha10 comprises a Doxorubicin derivative which is 3′-deamino-3″-4′-anhydro-[2″(S)-methoxy-3″(R)-hydroxy-4″-morpholinyl]doxorubicin.

In some embodiments, the cytotoxic moiety is a chemotherapeutic agent. For example, in some embodiments the chemotherapeutic agent may be an alkylating agent, an antimetabolite, an anti-microtubule agent, a topoisomerase inhibitor or a cytotoxic antibiotic. For example, in some embodiments the chemotherapeutic agent may be selected from the group consisting of Anthracyclines, Taxanes and Platinum agents. For example, in some embodiments the chemotherapeutic agent may be selected from the group consisting of Cisplatin, Paclitaxel, albumin-bound Paclitaxel, Docetaxel, Cyclophosphamide, Eribulin, Epirubicin, Doxorubicin, Carboplatin, Gemcitabine, Bleomycin, Fluorouracil, Cyclophosphamide, Vinorelbine, Capecitabine, Ixabepilone and Ixabepilone, or combinations thereof.

In some embodiments, the transcription toxin is selected from the group consisting of shiga and shiga-like toxins; type I ribosome inactivating proteins, type II ribosome inactivating proteins and saporin, or combinations thereof.

In some embodiments, the type I ribosome inactivating protein is trichosanthin and/or luffin.

In some embodiments, the type II ribosome inactivating protein is ricin, agglutinin and/or abrin.

In some embodiments, the cytotoxic drug is suitable for use in activation therapy, such as photon activation therapy, neutron activation therapy, neutron induced Auger electron therapy, synchrotron irradiation therapy, or low energy X-ray photon activation therapy.

In some embodiments, the antibody or antigen-binding fragment thereof with binding specificity for integrin alpha10 comprises a detectable moiety.

In some embodiments, the detectable moiety is selected from the group consisting of a fluorophore, an enzyme and a radioactive tracer or radioisotope.

In some embodiments, the detectable moiety comprises or consists of a radioisotope.

In some embodiments, the radioisotope is selected from the group consisting of ^{99m}Tc, ¹¹¹In, ⁶⁷Ga, ⁶⁸Ga, ⁷²As, ⁸⁹Zr, ¹²³I and ²⁰¹Tl.

In some embodiments, the antibody or antigen-binding fragment thereof with binding specificity for integrin alpha10 comprises a pair of detectable and cytotoxic radioisotopes, such as ⁸⁶Y/⁹⁰Y or ¹²⁴I/²¹¹At.

In some embodiments, the antibody or antigen-binding fragment thereof with binding specificity for integrin alpha10 comprises a radioisotope which is capable of simultaneously acting in a multi-modal manner as a detectable moiety and as a cytotoxic moiety.

In some embodiments, the detectable moiety comprises or consists of a paramagnetic isotope.

In some embodiments, the paramagnetic isotope is selected from the group consisting of ¹⁵⁷Gd, ⁵⁵Mn, ¹⁶²Dy, ⁵²Cr and ⁵⁶Fe.

In some embodiments, the detectable moiety is detectable by an imaging technique such as SPECT, PET, MRI, optical or ultrasound imaging.

In some embodiments, the cytotoxic moiety and/or detectable moiety is joined to the antibody or antigen-binding fragment thereof indirectly, via a linking moiety.

In some embodiments, the linking moiety is a chelator.

In some embodiments, the chelator is selected from the group consisting of derivatives of 1,4,7,10-tetraazacyclododecane-1,4,7,10,tetraacetic acid (DOTA), deferoxamine (DFO), derivatives of diethylenetriaminepentaacetic avid (DTPA), derivatives of S-2-(4-Isothiocyanatobenzyl)-1,4,7-triazacyclononane-1,4,7-triacetic acid (NOTA) and derivatives of 1,4,8,11-tetraazacyclodocedan-1,4,8,11-tetraacetic acid (TETA).

In some embodiments, the antibody or antigen-binding fragment thereof with binding specificity for integrin alpha10 does not comprise a cytotoxic moiety or a detectable moiety.

### Production of the Antibody

Another aspect of the invention relates to a polynucleotide encoding the antibody or antigen-binding fragment of the first aspect of the invention, or a component polypeptide chain thereof.

By “polynucleotide” we include DNA (e.g. genomic DNA or complementary DNA) and mRNA molecules, which may be single- or double-stranded.

In some embodiments, the polynucleotide is an isolated polynucleotide.

In some embodiments, the polynucleotide is a cDNA molecule.

It will be appreciated by persons skilled in the art that the polynucleotide may be codon-optimised for expression of the antibody or antigen-binding fragment in a particular host cell, e.g. for expression in human cells (for example, see Angov, 2011, Biotechnol. J. 6(6):650-659, the disclosures of which are incorporated herein by reference).

In some embodiments, the polynucleotide encoding the antibody or antigen-binding fragment of the invention is encoding an antibody light chain or variable region thereof.

In some embodiments, the polynucleotide encoding the antibody or antigen-binding fragment of the invention is encoding an antibody heavy chain or variable region thereof.

Another aspect of the invention relates to a vector comprising the polynucleotide according to another aspect of the invention.

In some embodiments, the vector is an expression vector.

The term “expression vector” is defined herein as a DNA molecule, for example linear or circular, that comprises a polynucleotide encoding a polypeptide of the present invention (the antibody or antigen-binding fragment thereof) and is operably linked to additional nucleotides that provide for its expression. The term “plasmid”, “expression vector” and “vector” are used interchangeably as the plasmid is the most commonly used form of vector at present. However the invention is intended to include such other forms of expression vectors that serve equivalent functions.

Another aspect of the invention relates to a recombinant host cell comprising the polynucleotide according to another aspect of the invention or a vector according to another aspect of the invention.

In some embodiments, the recombinant host cell is a bacterial cell.

In some embodiments, the recombinant host cell is a yeast cell.

In some embodiments, the recombinant host cell is a mammalian cell.

In some embodiments, the recombinant host cell is a human cell.

Another aspect of the invention relates to a method for producing the antibody or antigen-binding fragment of the another aspect of the invention, the method comprising culturing the host cell of another aspect of the invention comprising the polynucleotide of another aspect of the invention or the vector of the third aspect of the invention, under conditions which permit expression of the encoded antibody or antigen-binding fragment thereof.

### Detection of Integrin Alpha10

Another aspect of the invention relates to an in vitro method for the detection of cells expressing integrin alpha10beta1 in a subject, the method comprising:
(a) providing a sample of cells from a subject to be tested, such as biopsy tissue or blood sample;
(b) optionally, extracting and/or purifying the cells present in the sample;
(c) contacting the antibody or antigen-binding fragment of the first aspect of the invention with cells present in the sample;
(d) determining whether the antibody or antigen-binding fragment thereof binds to the cells
   wherein the binding of the antibody or antigen-binding fragment thereof to the cells is indicative of the presence of a disease or disorder associated with cells expressing integrin alpha10beta1 in the tissue of a subject.

In some embodiments, the antibody is covalently bound to a detectable moiety, such as a detectable moiety selected from the group consisting of a fluorophore, an enzyme, a radioactive tracer or a radioisotope. The integrin alpha10 antigen may also be detected by detecting a peptide, protein or polypeptide other than integrin alpha10 polypeptide, wherein said other peptide, protein or polypeptide is capable of specifically binding to an integrin alpha10 antigen. In some embodiments said peptide, protein or polypeptide is linked to an enzyme, a fluorophore or a radioactive tracer. The radioactive tracer may e.g. be selected from a positron emitter, or a gamma emitter. Conjugation of the antibody to a detectable moiety facilitates and improves detection of said antibody, which in turn may facilitate detection of integrin alpha10-expressing cells in a sample and so the diagnosis of a cancer.

In some embodiments, the antibody of the present disclosure can be used to detect integrin alpha10 on cells, in tissues, in blood of a sample obtained from a mammal, such as in vitro, or even in vivo and/or in situ by using in vivo antibody-based detection techniques described herein and/or known to the person of skill in art.

The person of skill in the art is capable of selecting the standard laboratory equipment for detection of the integrin alpha10 antibodies, depending on the situation and physical state of the sample.

In some embodiments, the person of skill in the art would conduct the detection step using flow cytometry such as Fluorescence-Activated Cell Sorting (FACS).

Typical immunological methods well known in the art include but are not limited to western blot, enzyme linked immunosorbent assay (ELISA), radioimmunoassay (RIA), immunohistochemistry (IHC), immunofluorescent assay (IF), fluorescence in situ hybridization (FISH).

Detecting integrin alpha10 can be achieved using methods well known in the art of detection and imaging, such as clinical imaging, such as conventional fluorescence microscopes, confocal microscope, 2-photon microscopes, stimulated emission depletion (STED) etc.

In some embodiments, the detectable moiety is selected from the group consisting of a fluorophore, an enzyme or a radioactive tracer.

Typical methods for detection of cell surface antigens in vivo are well known in the art include but are not limited to fluorescence imaging, positron emission tomography, x-ray computed tomography (CT), magnetic resonance imaging (MRI) and functional magnetic resonance imaging (fMRI), ultrasound and single-photon emission computed tomography (SPECT). In particular cell surface antigens can be imaged in vivo using immunolabeling with a radioactive tracer bound to an antibody or other specifically binding protein.

In some embodiments, the antibodies used for in vivo imaging are antibody fragments such as Fab fragments, and single chain antibodies due to their smaller size and absence of effector function.

### Pharmaceutical Compositions and Administration Thereof

Another aspect of the invention relates to a pharmaceutical composition comprising an effective amount of:
the antibody or antigen-binding fragment of another aspect of the invention,
the polynucleotide of another aspect of the invention,
the vector of another aspect of the invention, and/or
the host cell of another aspect of the invention,
in a pharmaceutical composition, wherein the composition further comprises a pharmaceutically-acceptable buffer, diluent, carrier or excipient.

In some embodiments, the present disclosure relates to compositions, such as pharmaceutical compositions, comprising:
an antibody specifically binding to an integrin alpha10 polypeptide or a fragment thereof, wherein said compositions are for use in the diagnosis and/or treatment of a cancer form as defined herein.

In some embodiments, the present disclosure relates to compositions, such as pharmaceutical compositions,
for use in the diagnosis and/or treatment of a cancer form selected from the group consisting of breast cancer, brain cancer, cancers of the CNS, lung cancer, prostate cancer, pancreatic cancer, skin cancer, lymphoma and sarcoma, or a metastasis of any one of said cancer forms. A detailed definition of the cancer forms which may be treated and/or diagnosed using the antibodies and compositions of the present disclosure is found elsewhere herein, for example in the section “Clinical conditions”.

In some embodiments, the composition for use in the diagnosis and/or treatment of a cancer form as defined herein comprises a pharmaceutically effective amount, such as a pharmacologically effective concentration of an antibody specifically binding to an integrin alpha10 polypeptide or a fragment thereof.

A pharmacologically effective concentration as referred to herein is typically a concentration of integrin alpha10 antibody, which induces the desired response in an individual receiving said pharmaceutical composition.

In some embodiments, the composition for use in the diagnosis and/or treatment of a cancer form as defined herein comprises a pharmacologically effective concentration of an antibody specifically binding to an integrin alpha10 polypeptide or a fragment thereof, wherein the antibody or fragment thereof is conjugated to an additional moiety.

For example, the additional moiety may be a detectable moiety. An antibody specifically binding to an integrin alpha10 polypeptide or a fragment thereof and conjugated to a detectable moiety may be useful in detecting integrin alpha10 expression on a cell and so determining that said cell may be a malignant cell and/or tumor-associated cell.

For example, the additional moiety may be a cytotoxic moiety. An antibody specifically binding to an integrin alpha10 polypeptide or a fragment thereof and conjugated to a cytotoxic moiety, such as an antibody drug conjugate (ADC) comprising an antibody specifically binding to an integrin alpha10 polypeptide or a fragment thereof, may be useful in specifically directing a certain cytotoxic moiety and/or drug to a cell expressing integrin alpha10 and being a malignant cell and/or tumor-associated cell.

An ADC can be generated using well-established conjugation approaches. For example, a target antibody may be conjugated to a payload of DNA intercalator and Topoisomerase Inhibitor type by mild reduction of interchain and/or intrachain disulfides bond.

For example, the additional moiety may comprise a biological response modifier. Biological response modifiers are substances that modify immune responses by either enhance an immune response or suppress it. Biological response modifiers may be endogenous, such as moieties usually produced naturally within the body, or exogenous.

In some embodiments the additional moiety may comprise a biological response modifier, such as a cytokine, a lymphokine, an interferon or combinations thereof.

The compositions for use of the present disclosure may be pharmaceutical compositions suitable for parenteral administration. Such compositions preferably include aqueous and non-aqueous sterile injection solutions which may contain wetting or emulsifying reagents, anti-oxidants, pH buffering agents, bacteriostatic compounds and solutes which render the formulation isotonic with the body fluid, preferably the blood, of the individual; and aqueous and non-aqueous sterile suspensions which may include suspending agents or thickening agents. The pharmaceutical composition may be presented in unit-dose or multi-dose containers, for example, sealed ampoules and vials and may be stored in a freeze-dried condition requiring only the addition of the sterile liquid carrier immediately prior to use.

In some embodiments, the composition for use of the present disclosure further comprises at least one pharmaceutically acceptable buffer, diluent, carrier or excipient.

Preferably, the composition of the present invention comprises one or more suitable pharmaceutical excipients, which could be non-sterile or sterile, for use with cells, tissues or organisms, such as pharmaceutical excipients suitable for administration to an individual. Such excipients may include, but are not limited to, saline, buffered saline, dextrose, water, glycerol, ethanol and combinations of these excipients in various amounts. The formulation should suit the mode of administration.

Preferably, the pharmaceutical compositions of the present invention are prepared in a form which is injectable, either as liquid solutions or suspensions; furthermore solid forms suitable for solution in or suspension in liquid prior to injection are also within the scope of the present invention. The preparation may be also be emulsified or encapsulated in liposomes.

The integrin alpha10 polypeptide antibody, or the polynucleotide specifically binding to a polynucleotide transcript which encodes an integrin alpha10 polypeptide, may be administered alone or in combination with other compounds, either simultaneously or sequentially in any order.

Administration could for example be parenteral via injection or infusion. Parenteral injection could for example be intraventricular, intrathecal, intratumoral, intravenous, intramuscular, intradermal or subcutaneous injection. Preferably, said administration is parenterally by injection or infusion.

It will be appreciated by persons skilled in the art that additional compounds may also be included in the pharmaceutical compositions, including, chelating agents such as EDTA, citrate, EGTA or glutathione.

The pharmaceutical compositions may be prepared in a manner known in the art that is sufficiently storage stable and suitable for administration to humans and animals. For example, the pharmaceutical compositions may be lyophilised, e.g. through freeze drying, spray drying, spray cooling, or through use of particle formation from supercritical particle formation.

The excipient may be one or more of carbohydrates, polymers, lipids and minerals. Examples of carbohydrates include lactose, glucose, sucrose, mannitol, and cyclodextrines, which are added to the composition, e.g. for facilitating lyophilisation. Examples of polymers are starch, cellulose ethers, cellulose carboxymethylcellulose, hydroxypropylmethyl cellulose, hydroxyethyl cellulose, ethylhydroxyethyl cellulose, alginates, carrageenans, hyaluronic acid and derivatives thereof, polyacrylic acid, polysulphonate, polyethyleneglycol/polyethylene oxide, polyethyleneoxide/polypropylene oxide copolymers, polyvinylalcohol/polyvinylacetate of different degree of hydrolysis, and polyvinylpyrrolidone, all of different molecular weight, which are added to the composition, e.g., for viscosity control, for achieving bioadhesion, or for protecting the lipid from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

The antibody or antigen-binding fragment of the invention may be formulated into any type of pharmaceutical composition known in the art to be suitable for the delivery thereof.

In some embodiments, the pharmaceutical compositions of the invention may be in the form of a liposome, in which the antibody or antigen-binding fragment is combined, in addition to other pharmaceutically acceptable carriers, with amphipathic agents such as lipids, which exist in aggregated forms as micelles, insoluble monolayers and liquid crystals. Suitable lipids for liposomal formulation include, without limitation, monoglycerides, diglycerides, sulfatides, lysolecithin, phospholipids, saponin, bile acids, and the like. Suitable lipids also include the lipids above modified by poly(ethylene glycol) in the polar headgroup for prolonging bloodstream circulation time. Preparation of such liposomal formulations is can be found in for example U.S. Pat. No. 4,235,871, the disclosures of which are incorporated herein by reference.

The pharmaceutical compositions of the invention may also be in the form of biodegradable microspheres. Aliphatic polyesters, such as poly(lactic acid) (PLA), poly(glycolic acid) (PGA), copolymers of PLA and PGA (PLGA) or poly(caprolactone) (PCL), and polyanhydrides have been widely used as biodegradable polymers in the production of microspheres. Preparations of such microspheres can be found in U.S. Pat. No. 5,851,451 and in EP 0 213 303, the disclosures of which are incorporated herein by reference.

In some embodiments, the pharmaceutical compositions of the invention are provided in the form of polymer gels, where polymers such as starch, cellulose ethers, cellulose carboxymethylcellulose, hydroxypropylmethyl cellulose, hydroxyethyl cellulose, ethylhydroxyethyl cellulose, alginates, carrageenans, hyaluronic acid and derivatives thereof, polyacrylic acid, polyvinyl imidazole, polysulphonate, polyethyleneglycol/polyethylene oxide, polyethyleneoxide/polypropylene oxide copolymers, polyvinylalcohol/polyvinylacetate of different degree of hydrolysis, and polyvinylpyrrolidone are used for thickening of the solution containing the agent. The polymers may also comprise gelatine or collagen.

Alternatively, the antibody or antigen-binding fragment may simply be dissolved in saline, water, polyethylene glycol, propylene glycol, ethanol or oils (such as safflower oil, corn oil, peanut oil, cottonseed oil or sesame oil), tragacanth gum, and/or various buffers.

It will be appreciated that the pharmaceutical compositions of the invention may include ions and a defined pH for potentiation of action of the active antibody or antigen-binding fragment. Additionally, the compositions may be subjected to conventional pharmaceutical operations such as sterilisation and/or may contain conventional adjuvants such as preservatives, stabilisers, wetting agents, emulsifiers, buffers, fillers, etc.

The pharmaceutical compositions according to the invention may be administered via any suitable route known to those skilled in the art. Thus, possible routes of administration include parenteral (intravenous, subcutaneous, and intramuscular), topical, ocular, nasal, pulmonar, buccal, oral, parenteral, vaginal and rectal. Also administration from implants is possible.

In one preferred embodiment, the pharmaceutical compositions are administered parenterally, for example, intravenously, intracerebroventricularly, intraarticularly, intra-arterially, intraperitoneally, intrathecally, intraventricularly, intrasternally, intracranially, intramuscularly or subcutaneously, or they may be administered by infusion techniques. They are conveniently used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Thus, the pharmaceutical compositions of the invention are particularly suitable for parenteral, e.g. intravenous, administration.

The pharmaceutical compositions will be administered to a patient in a pharmacologically effective dose.

In the context of diagnostic use of the antibody or antigen-binding fragment of the invention, a ‘pharmaceutically effective amount’, or ‘pharmacologically effective concentration’, or ‘effective concentration’, or ‘effective amount’, or ‘diagnostically effective’, as used herein, refers to that amount which provides a detectable signal for diagnosis, e.g. for in vivo imaging purposes.

It will be appreciated by persons skilled in the art that the pharmaceutical compositions of the invention may be administered alone or in combination with other therapeutic agents used in the treatment of neoplastic disorders or diseases.

In some embodiments, said composition is adapted for parenteral delivery.

In some embodiments, said composition is adapted for intravenous delivery.

In some embodiments, said composition is adapted for topical delivery.

In some embodiments, said composition is adapted for subcutaneous delivery.

In some embodiments, said composition is adapted for intramuscular delivery.

It will be further appreciated by persons skilled in the art that the antibody or antigen-binding fragment and pharmaceutical compositions or formulations of the present invention have utility in both the medical and veterinary fields. Thus, the methods of the invention may be used in the treatment of both human and non-human animals (such as horses, dogs and cats). Preferably, however, the patient is human.

### Indications and Use of the Antibody

Another aspect of the invention relates to
the antibody or antigen-binding fragment of another aspect of the invention,
the polynucleotide of another aspect of the invention,
the vector of another aspect of the invention,
the host cell of another aspect of the invention, and/or
the composition of another aspect of the invention,
for use in medicine.

Another aspect of the invention relates to
the antibody or antigen-binding fragment of another aspect of the invention,
the polynucleotide of another aspect of the invention,
the vector of another aspect of the invention,
the host cell of another aspect of the invention, and/or
the composition of another aspect of the invention,
for use in the prevention and/or treatment and/or alleviation and/or detection and/or diagnosis of a disease or disorder susceptible to treatment with an inhibitor of integrin alpha10beta1, and/or
wherein the disease or disorder is associated with cells expressing integrin alpha10beta1.

Depending upon whether it is therapeutically desirable to kill the target cells expressing integrin alpha10beta1, for example in the case of neoplastic cells, an antibody or antigen-binding fragment according to the first aspect of the invention may be used that it capable of inducing ADCC and/or ADCP and/or CDC. For example, where the target cells expressing integrin alpha10beta1 are cancer cells it may be advantageous for the antibody or antigen-binding fragment to be capable of inducing ADCC in order to eliminate such cells.

In one embodiment, the antibody or antigen-binding fragment of the present disclosure activates an ADCC response.

However, it will be appreciated that a therapeutic benefit may also be achieved using an antibody or antigen-binding fragment that lacks ADCC activity.

In an aspect of the invention, the invention relates to
the antibody or antigen-binding fragment of another aspect of the invention,
the polynucleotide of another aspect of the invention,
the vector of another aspect of the invention,
the host cell of another aspect of the invention, and/or
the composition of another aspect of the invention,
for use in the prevention and/or treatment and/or alleviation and/or detection and/or diagnosis of a disease or disorder susceptible to treatment with an inhibitor to integrin alpha10beta1.

In an aspect of the invention, the invention relates to the antibody or antigen-binding fragment of another aspect of the invention,
the polynucleotide of another aspect of the invention,
the vector of another aspect of the invention,
the host cell of another aspect of the invention, and/or
the composition of another aspect of the invention,
for use in the prevention and/or treatment and/or alleviation and/or detection and/or diagnosis of a disease or disorder, wherein the disease or disorder is associated with cells expressing integrin alpha10beta1.

In some embodiments, said disease or disorder is associated with integrin alpha10beta1 signaling.

In relation to the therapeutic and prophylactic aspects of the invention, it will be appreciated by persons skilled in the art that binding of the antibody, or antigen binding fragment thereof, to integrin alpha10 present on the surface of the cells associated with the neoplastic disease or disorder may lead to a modulation (i.e. an increase or decrease) of a biological activity of integrin alpha10beta1. However, such a modulatory effect is not essential; for example, the antibody or antigen binding fragment thereof of the invention may elicit a therapeutic and prophylactic effect simply by virtue of binding to integrin alpha10 on the surface of the cells associated with the disease or disorder, which in turn may trigger the immune system to induce processes, such as cell death (e.g. by ADCC and/or by the presence within the agent of a cytotoxic/radioactive moiety).

In some embodiments, the antibody or antigen-binding fragment thereof with binding specificity for integrin alpha10 inhibits the biological activity of integrin alpha10beta1.

By “biological activity of integrin alpha10beta1” we include any interaction or signaling event which involves integrin alpha10beta1 on the cells associated with the neoplastic disease or disorder. Some biological activities, which may be inhibited by the antibody, antigen or fragments thereof of the present disclosure, and which are associated with cells associated with a neoplastic disease or disorder, are cell adhesion, cell proliferation, cell migration, cell survival, tumour growth and metastasis, integrin alpha10beta1 signalling and/or any combination thereof. These are described herein in the section “Inhibition of biological activities”.

Another aspect of the invention relates to the antibody or antigen-binding fragment of another aspect of the invention,
the polynucleotide of another aspect of the invention,
the vector of another aspect of the invention,
the host cell of another aspect of the invention, and/or
the composition of another aspect of the invention,
for use in inhibiting cell survival, cell migration, cell proliferation, cell growth and/or cell adhesion, tumour growth and metastasis, wherein targeted cells express integrin alpha10beta1.

Another aspect of the invention relates to a method for in vivo imaging the expression of the integrin alpha10beta1 in a mammal, the method comprising the steps of
a) Providing a mammal,
b) Providing an antibody or antigen-binding fragment thereof as disclosed herein,
c) administering the antibody or antigen-binding fragment thereof to the mammal so as to allow the antibody or a fragment thereof to bind to an extracellular domain of integrin alpha10beta1 of cells in said mammal,
d) optionally adding a second labelled antibody or a fragment thereof to the sample, wherein the second antibody or a fragment thereof binds to the antibody or a fragment thereof in c),
e) detecting the antibody or antigen-binding fragment thereof on said cells in c), or optionally detecting the second labelled antibody or a fragment thereof in d) bound to the antibody or a fragment thereof, and
   creating an image of the detected antibody or a fragment thereof, thereby imaging the expression of integrin alpha10beta1 on cells in a mammal in vivo.

In vitro diagnostic assays based on an antibody or antigen-binding fragment thereof as disclosed herein can be used to detect the presence of integrin alpha10 or a fragment thereof on the surface of extracellular vesicles, such as exosomes, which have left the tumour site; such assays can also be used to detect the presence of integrin alpha10 or a fragment thereof that is no longer attached to a cell or vesicle, for example in case of shredding of cells, such as tumour cells. For example, an antibody or antigen-binding fragment thereof as disclosed herein can be used to detect the presence of integrin alpha10 on the surface of extracellular vesicles, such as exosomes, as well as fragments of integrin alpha10 as found in blood and/or plasma.

Thus, another aspect of the invention relates to a method for in vitro detection of integrin alpha10 or a fragment thereof in a sample obtained from a mammal, the method comprising the steps of
a) Providing a sample obtained from a mammal,
b) Providing an antibody or antigen-binding fragment thereof as disclosed herein,
c) Contacting the antibody or antigen-binding fragment thereof with the sample so as to allow the antibody or a fragment thereof to bind to an extracellular domain of integrin alpha10, or a fragment thereof, in said sample,
d) optionally adding a second labelled antibody or a fragment thereof to the sample, wherein the second antibody or a fragment thereof binds to the antibody or a fragment thereof in c),
e) detecting the antibody or antigen-binding fragment thereof, or optionally detecting the second labelled antibody or a fragment thereof in d) bound to the antibody or a fragment thereof, and
   thereby determining whether integrin alpha10 or a fragment thereof is found in the sample obtained from the mammal.

For example, the sample can be a mammalian body fluid, such as blood, plasma, cerebrospinal fluid, urine.

In some embodiments, the sample comprises cells, extracellular vesicles and/or fragments thereof.

### Clinical Conditions

In some embodiments, the cancer form to be treated and/or prevented and/or detected and/or diagnosed and/or classified and/or determined a prognosis for and/or prevented from metastasizing is selected from the group consisting of breast cancer, brain cancer, cancers of the central nervous system (CNS), lung cancer, prostate cancer, pancreatic cancer, skin cancer, lymphoma, and sarcoma.

In some embodiments, the breast cancer is a triple negative breast cancer form, and said triple negative breast cancer is selected from the group consisting of basal-like 1 breast cancer, basal-like 2 breast cancer, claudin-low breast cancer, metaplastic breast cancer (MBC), interferon-rich breast cancer, immunomodulatory breast cancer, mesenchymal breast cancer, mesenchymal stem-like breast cancer, luminal androgen receptor breast cancer and unstable breast cancer.

In some embodiments, the breast cancer is a triple negative breast cancer form and it has morphological features of invasive ductal carcinoma.

In some embodiments, the breast cancer is a triple negative breast cancer form and it has morphological features of basal-like triple negative breast cancer.

In some embodiments, the breast cancer is a basal-like breast cancers form and it has morphological features of invasive ductal carcinoma.

In some embodiments, the cancer is prostate cancer, and the prostate cancer is small cell (neuroendocrine) carcinoma (SCNC) or castrate-resistant prostate cancer (CRPC).

In some embodiments, the cancer is lung cancer, and the lung cancer is squamous cell lung carcinoma, lung adenocarcinoma, small-cell lung carcinoma or large cell lung carcinoma.

In some embodiments, the cancer is a pancreas cancer, and the pancreas cancer is an exocrine tumor or an endocrine tumor.

In some embodiments the pancreas cancer is an exocrine tumor selected from the group consisting of ductal adenocarcinoma, acinar cell carcinoma, adeno-squamous carcinoma, intraductal papillary mucinous neoplasm (IPMN) and Pancreatic intraepithelial neoplasia.

In some embodiments the pancreas cancer is an endocrine tumor selected from the group consisting of neuroendocrine tumor, gastrinoma, glucagonoma, insulinoma, somatostatinoma, VIPoma, and non-functional Islet cell tumor In some embodiments, the cancer is pancreatic cancer, and the pancreatic cancer is wherein the pancreatic cancer is a neuroendocrine tumor.

In some embodiments, the cancer is pancreatic cancer, and the pancreatic cancer is a grade I, grade II or grade III pancreatic cancer.

In some embodiments, the sarcoma is selected from the group consisting of chondrosarcoma, osteosarcoma, undifferentiated pleomorphic sarcoma, myxofibrosarcoma, dedifferentiated liposarcoma, atypical lipomatous tumor, myxoinflammatory fibroblastic sarcoma, low grade fibromyxoid sarcoma, sclerosing epithelioid fibrosarcoma, pseudimyogenic hemangioendothelioma and mesenchymal chondrosarcoma.

In some embodiments, the brain cancer and/or the cancer of the CNS is selected from the group consisting of:
a) Tumours of neuroepithelial tissue selected from
   i) Astrocytic tumours selected from
   Pilocytic astrocytoma, Pilomyxoid astrocytoma, Subependymal giant cell astrocytoma, Pleomorphic xanthoastrocytoma, Diffuse astrocytoma, Anaplastic astrocytoma, Glioblastoma, Giant cell glioblastoma, Gliosarcoma, Gliomatosis cerebri, and
   ii) Oligodendroglial tumours selected from Oligodendroglioma and Anaplastic oligodendroglioma, and
   iii) Oligoastrocytic tumours selected from Oligoastrocytoma and Anaplastic oligoastrocytoma, and
   iv) Ependymal tumours selected from Subependymoma, Myxopapillary ependymoma, Ependymoma, Anaplastic ependymoma, and
   v) Choroid plexus tumours selected from Choroid plexus papilloma, Atypical choroid plexus papilloma, and Choroid plexus carcinoma, and
   vi) Other neuroepithelial tumours selected from Astroblastoma, Choroid glioma of the third ventricle, and Angiocentric glioma and,
   vii) Neuronal and mixed neuronal-glial tumours selected from Dysplastic gangliocytoma of cerebellum (Lhermitte-Duclos), Desmoplastic infantile astrocytoma/ganglioglioma, Dysembryoplastic neuroepithelial tumour, Gangliocytoma, Ganglioglioma, Anaplastic ganglioglioma, Central neurocytoma, Extraventricular neurocytoma, Cerebellar liponeurocytoma,
   Papillary glioneuronal tumour, Rosette-forming glioneuronal tumour of the fourth ventricle, and Paraganglioma, and
   viii) Tumours of the pineal region selected from Pineocytoma, Pineal parenchymal tumour of intermediate differentiation, Pineoblastoma, and Papillary tumours of the pineal region, and
   ix) Embryonal tumours selected from Medulloblastoma, Medulloblastoma with extensive nodularity, Anaplastic medulloblastoma, CNS Primitive neuroectodermal tumour, CNS Neuroblastoma, and Atypical teratoid/rhabdoid tumour), and
b) Tumours of cranial and paraspinal nerves selected from
   i) Schwannoma, ii) Neurofibroma, iii) Perineurioma, and iv) Malignant peripheral nerve sheath tumour (MPNST), and
c) Tumours of the meninges selected from
   i) Tumours of meningothelial cells, selected from Meningioma, Atypical meningioma, Anaplastic meningioma,
   ii) Mesenchymal tumours selected from Lipoma, Angiolipoma, Hibernoma, Liposarcoma, Solitary fibrous tumour, Fibrosarcoma, Malignant fibrous histiocytoma, Leiomyoma, Leiomyosarcoma, Rhabdomyoma, Rhabdomyosarcoma, Chondroma, Chondrosarcoma, Osteoma, Osteosarcoma, Osteo-chondroma, Haemangioma, Epithelioid hemangioendothelioma, Haemangiopericytoma, Anaplastic haemangiopericytoma, and Angiosarcoma, Kaposi Sarcoma, Ewing Sarcoma—PNET,
   iii) Primary melanocytic lesions selected from
   Diffuse melanocytosis, Melanocytoma, Malignant melanoma, Meningeal melanomatosis, and
   iv) Other neoplasms related to the meninges such as Haem-angioblastoma, and
d) Tumours of the haematopoietic system selected from
   i) Malignant Lymphomas, Plasmocytoma, and ii) Granulocytic sarcoma, and
e) Tumours of the sellar region selected from
   i) Craniopharyngioma, ii) Granular cell tumour, iii) Pituicytoma, and iv) Spindle cell oncocytoma of the adenohypophysis.

The above list of CNS tumors is based on the more detailed, complete and updated classification of CNS tumors is found in Louis et al. (2021) The WHO Classification of Tumours of the Nervous System: a summary. Neuro-Oncology, Volume 23, Issue 8, August 2021, Pages 1231-1251.

In some embodiments the brain cancer and/or the cancer of the CNS is a glioma.

In some embodiments the brain cancer and/or the cancer of the CNS is a grade II, III or IV glioma.

In some embodiments the brain cancer and/or the cancer of the CNS is an astrocytoma, such as astrocytoma grade II, astrocytoma grade III or astrocytoma grade IV.

In some embodiments the glioma is a glioblastoma.

In some embodiments the glioma is primary glioblastoma.

In some embodiments the glioma is secondary glioblastoma.

In some embodiments the brain cancer and/or the cancer of the CNS is medulloblastoma.

In some embodiments the brain cancer and/or the cancer of the CNS is neuroblastoma.

In some embodiments the brain cancer and/or the cancer of the CNS is selected from the group consisting of astrocytomas, anaplastic astrocytomas, hemangiopericytomas of the brain, meningiomas, angiomatous hemangiomas, atypical meningiomas, fibroblastic meningiomas, meningiothelial meningiomas, secretory meningiomas, oligoastrocytomas, anaplastic oligoastrocytomas, oligodendrogliomas, and anaplastic oligodendrogliomas.

In some embodiments the brain cancer and/or the cancer of the CNS is selected from the group consisting of Astrocytic tumours, Oligodendroglial tumours, Ependymal cell tumours, Mixed gliomas, Neuroepithelial tumours of uncertain origin, Tumours of the choroid plexus, Neuronal and mixed neuronal-glial tumours, Pineal Parenchyma Tumours and Tumours with neuroblastic or glioblastic elements (embryonal tumours), ependymomas, astrocytomas, oligodendrogliomas, oligoastrocytomas, neuroepithelial tumours, and neuronal and mixed neuronal-glial tumours.

In some embodiments, the cancer is a metastasis, for example, it may be a metastasis of any one of breast cancer, brain cancer, cancers of the CNS, lung cancer, prostate cancer, pancreatic cancer, skin cancer, lymphoma and sarcoma.

In some embodiments, the cancer is a metastasis of any one of triple negative breast cancer and inflammatory breast cancer.

In some embodiments, the cancer is a metastasis of triple negative breast cancer, such as of a triple negative breast cancer selected from the group consisting of basal-like 1 breast cancer, basal-like 2 breast cancer, claudin-low breast cancer, metaplastic breast cancer (MBC), interferon-rich breast cancer, immunomodulatory breast cancer, mesenchymal breast cancer, mesenchymal stem-like breast cancer, luminal androgen receptor breast cancer and unstable breast cancer.

In some embodiments, the cancer is a metastasis of a lung cancer. In some embodiments, the cancer form is a metastasis of squamous cell lung carcinoma, lung adenocarcinoma, small-cell lung carcinoma or large cell lung carcinoma.

In some embodiments, the cancer is a metastasis of a pancreatic cancer.

In some embodiments, the cancer is a metastasis of a prostate cancer.

In some embodiments, the cancer is a metastasis of a sarcoma.

In some embodiments, the cancer is a metastasis of a brain cancer and/or the cancer of the CNS.

In some embodiments, the cancer is a metastasis of a brain cancer and/or a cancer of the CNS selected from the group consisting of astrocytomas, anaplastic astrocytomas, hemangiopericytomas of the brain, meningiomas, angiomatous hemangiomas, atypical meningiomas, fibroblastic meningiomas, meningiothelial meningiomas, secretory meningiomas, oligoastrocytomas, anaplastic oligoastrocytomas, oligodendrogliomas, and anaplastic oligodendrogliomas.

### Treatment of Cancer

The person skilled in the art will recognize that the antibody or antigen-binding fragment thereof with binding specificity for integrin alpha10 disclosed by some embodiments of the present invention can be used to treat cancer.

In some embodiments, the antibody or antigen-binding fragment thereof has binding specificity for integrin alpha10 and thus binds cells expressing integrin alpha10beta1, which are: malignant cells and/or tumor-associated cells, for example cancer associated fibroblast (CAFs), stromal cells, stem cells and/or stem-like cells, and/or for example tumor-associated macrophages (TAMs), immune cells, endothelial cells.

In some embodiments, the antibody or antigen-binding fragment thereof with binding specificity for integrin alpha10 is for use in treating cancer, wherein a treatment is initiated upon detection of an integrin alpha10 polypeptide in a cancer cell in a tumor of a subject.

In some embodiments, the antibody or antigen-binding fragment thereof with binding specificity for integrin alpha10 is administered to an individual in need thereof in combination with radiation therapy and/or surgical removal of cancer, such as prior to radiation therapy and/or surgical removal of cancer, such as after radiation therapy and/or surgical removal of cancer.

Another aspect of the invention relates to the antibody or antigen-binding fragment of the first aspect of the invention for use in inducing cell death and/or inhibiting the growth and/or proliferation of pathological cells associated with a neoplastic disorder in a subject, or stem cells or progenitor cells thereof, wherein the cells express integrin alpha10beta1.

Another aspect of the invention relates to the use of
the antibody or antigen-binding fragment of another aspect of the invention,
the polynucleotide of another aspect of the invention,
the vector of another aspect of the invention,
the host cell of another aspect of the invention, and/or
the composition of another aspect of the invention,
in the preparation of a medicament for the prevention, treatment, alleviation, detection and/or diagnosis of a disease or disorder susceptible to treatment with an inhibitor of integrin alpha10 signaling,
and/or wherein the disease or disorder is associated with cells expressing integrin alpha10beta1.

Similarly, another aspect of the invention relates to a method for the prevention and/or treatment and/or alleviation and/or detection and/or diagnosis of a disease or disorder susceptible to treatment with an inhibitor of integrin alpha10beta1 signaling and/or wherein the disease or disorder is associated with cells expressing integrin alpha10 in a subject, comprising the step of administering to the subject an effective amount of
the antibody or antigen-binding fragment of another aspect of the invention,
the polynucleotide of another aspect of the invention,
the vector of another aspect of the invention,
the host cell of another aspect of the invention, and/or
the composition of another aspect of the invention.

Another aspect of the invention concerns the use of a composition comprising:
an antibody specifically binding to an integrin alpha10 polypeptide or a fragment thereof, or
a polynucleotide specifically binding to a polynucleotide transcript which encodes an integrin alpha10 polypeptide or a fragment or variant thereof,
for the manufacture of a medicament for the treatment of a cancer, wherein said cancer is a cancer expressing integrin alpha10, such as wherein said cancer is selected from the group consisting of breast cancer, brain cancer, cancers of the CNS, lung cancer, prostate cancer, pancreatic cancer, skin cancer, lymphoma and sarcoma, or wherein said cancer is a metastasis.

Another aspect of the present disclosure relates to a method of treating a cancer, wherein said cancer is selected from the group consisting of breast cancer, brain cancer, cancers of the CNS, lung cancer, prostate cancer, pancreatic cancer, skin cancer, lymphoma and sarcoma, or wherein said cancer is a metastasis, the method comprising administering a pharmaceutically effective amount of a composition comprising:
an antibody specifically binding to an integrin alpha10 polypeptide or a fragment thereof, or
a polynucleotide specifically binding to a polynucleotide transcript which encodes an integrin alpha10 polypeptide or a fragment or variant thereof, to a subject in the need thereof.

Another aspect of the invention relates to a method for treating a patient with a disease or disorder associated with cells expressing integrin alpha10beta1, the method comprising:
a) Selecting a patient identified as having a disease or disorder associated with cells expressing integrin alpha10beta1 using a method according to another aspect of the invention; and
b) Administering to said patient a therapeutic agent effective in the treatment of said disease or disorder.

Another aspect of the present disclosure relates to a method of inhibiting integrin alpha10beta1-mediated signaling of at least one cancer cell, the method comprising contacting the at least one cancer cell with a composition comprising an effective amount of:
a) an antibody or antigen-binding fragment specific for integrin alpha10 polypeptide; and/or
b) a polynucleotide transcript which encodes an integrin alpha10 polypeptide or a fragment or variant thereof,
   wherein the at least one cancer cell is selected from the group consisting of a breast cancer cell, a brain cancer cell, a cancer cell of a cancer of the CNS, a lung cancer cell, a prostate cancer cell, a pancreatic cancer cell, skin cancer cell, a lymphoma cell, a sarcoma cell and a metastatic tumor cell, or a cancer cells of any of the cancer types listed herein under “Clinical conditions”.

It is understood by the person skilled in the art that cellular signaling includes molecular mechanisms whereby cells detect and respond to external stimuli. Cell signaling also includes transcriptional and translational controls and mechanisms as well as signal transduction mechanisms.

One aspect of the present disclosure relates to a method of inhibiting cellular functions of at least one cancer cell, the method comprising contacting the at least one cancer cell with an effective amount of a composition comprising:
an antigen comprising an integrin alpha10 polypeptide or a fragment thereof; and/or a polynucleotide transcript which encodes an integrin alpha10 polypeptide or a fragment or variant thereof,
wherein the at least one cancer cell is selected from the group consisting of a breast cancer cell, a brain cancer cell, a cancer cell of a cancer of the CNS, a lung cancer cell, a prostate cancer cell, a pancreatic cancer cell, a skin cancer cell, a lymphoma cell, a sarcoma cell and a metastatic tumor cell, or a cancer cells of any of the cancer types listed herein under “Clinical conditions”.

In some embodiments, the present invention relates to a method for inhibiting the growth and/or proliferation of a cell expressing integrin alpha10beta1 comprising administering a composition comprising an effective amount of:
an antibody specifically binding to an integrin alpha10 polypeptide or a fragment thereof, or
a polynucleotide specifically binding to a polynucleotide transcript which encodes an integrin alpha10 polypeptide or a fragment or variant thereof.

In some embodiments of the present disclosure, inhibiting at least one cancer cell is selected from the group consisting of:
inhibiting proliferation of the at least one cancer cell;
inhibiting growth of the at least one cancer cell;
inhibiting self-renewal of the at least one cancer cell;
inhibiting anchorage-independent growth of the at least one cancer cell;
inhibiting migration of the at least one cancer cell;
inhibiting invasion of the at least one cancer cell;
inhibiting survival of the at least one cancer cell;
inhibiting adhesion of the at least one cancer cell; and/or
combinations thereof.

In another embodiment, inhibiting at least one cancer cell by i) an antibody specifically binding to an integrin alpha10 polypeptide or ii) a polynucleotide specifically binding to a polynucleotide transcript which encodes an integrin alpha10 polypeptide or a fragment or variant thereof, inhibits anchorage-dependent growth of the at least one cancer cell.

In some embodiments, the present disclosure relates to a method of inhibiting at least one cancer cell, wherein the cancer cell is a metastatic tumor, and wherein said inhibiting is at least one of:
survival of the metastatic tumor;
growth of the metastatic tumor;
proliferation of the metastatic tumor;
migration of the metastatic tumor;
invasion of the metastatic tumor;
initiation of new metastatic tumors;
infiltration of new metastatic tumors; and
combinations thereof.

The cell may express one or more further markers as defined herein. In some embodiments, said cell is a malignant cells and/or tumor-associated cell. In another embodiment said cell is a cancer associated fibroblast (CAFs), a stromal cell, a stem cells and/or a stem-like cell. Said method may be performed in vitro or in vivo.

In some embodiments, the composition comprising an effective amount of:
an antibody specifically binding to an integrin alpha10 polypeptide or a fragment thereof, or
a polynucleotide specifically binding to a polynucleotide transcript which encodes an integrin alpha10 polypeptide or a fragment or variant thereof, is capable of inducing cell death and/or inhibiting the growth and/or inhibiting proliferation and/or inhibiting migration of cells expressing an integrin alpha10.

In some embodiments, the treatment is initiated upon detection of an integrin alpha10 polypeptide and/or polynucleotide transcript in a cancer cell in a tumor of said subject.

In some embodiments, the methods disclosed herein target an antigen comprising an integrin alpha10 polypeptide or a fragment thereof that is expressed on the surface of the cells.

One aspect of the present disclosure relates to a method of preventing metastasis from a primary cancer form selected from the group consisting of breast cancer, brain cancer, cancer of the CNS, lung cancer, prostate cancer, pancreatic cancer, skin cancer, lymphoma and sarcoma, or any of the cancer types listed herein under “Clinical conditions”, the method comprising administering a therapeutically effective amount of:
a) an antibody or antigen-binding fragment thereof, wherein the antibody or antigen binding fragment is specific for integrin alpha10 polypeptide; and/or
b) a polynucleotide transcript which encodes an integrin alpha10 polypeptide or a fragment or variant thereof,
   to a patient in need thereof.

It will be appreciated by the person skilled in the art that a method of preventing metastasis from a primary cancer can be administered upon detection of the primary cancer.

### Detection and Diagnosis of Cancer Forms

One aspect of the present disclosure relates to an agent comprising or consisting of an antibody with specificity for an integrin alpha10 polypeptide or a fragment thereof, for use in detecting cells associated with a cancer form of a mammal, wherein the cells express an integrin alpha10 polypeptide, and wherein said cancer form is selected from the group consisting of breast cancer, brain cancer, cancer of the CNS, lung cancer, prostate cancer, pancreatic cancer, skin cancer, lymphoma and sarcoma, or a metastasis of any one of said cancer forms.

Another aspect of the invention relates to an in vitro method for identifying a patient with a disease or disorder associated with cells expressing integrin alpha10beta1 who would benefit from treatment with the antibody or antigen-binding fragment of the first aspect of the invention, the method comprising:
a) providing a sample, such as biopsy tissue or blood sample, from a patient to be tested;
b) optionally, extracting and/or purifying the cells present in the sample;
c) contacting an antibody or antigen-binding fragment thereof as defined herein with the sample;
d) determining whether the antibody or antigen-binding fragment thereof binds to an integrin alpha10 domain or fragment thereof,
   wherein the binding of the antibody or antigen-binding fragment thereof to an integrin alpha10 domain or fragment thereof is indicative of a patient who would benefit from treatment with an antibody or antigen-binding fragment thereof as defined herein.

In fact, cells expressing integrin alpha10beta1, such as cancer cells expressing integrin alpha10beta1, may undergo shedding, and/or degradation, for example due to necrosis of the tumour tissue or as part of their life cycle, and these may results in parts of the cells and proteins expressed on their surface to end up in the blood stream. Thus, cancer cells or cancer-associated cells expressing integrin alpha10 may be degraded and/or shed and integrin alpha10 or fragments thereof may end up in blood and be detected according to a method of the present disclosure by binding to an antibody of the present disclosure.

Another aspect of the invention relates to an in vivo method for identifying a patient with a disease or disorder associated with cells expressing integrin alpha10beta1 who would benefit from treatment with the antibody or antigen-binding fragment of the first aspect of the invention, the method comprising:
(a) contacting the antibody or antigen-binding fragment of the first aspect of the invention with cells present in the sample;
(b) determining whether the antibody or antigen-binding fragment thereof binds to the cells
   wherein the binding of the antibody or antigen-binding fragment thereof to cells expressing integrin alpha10beta1 is indicative of a patient who would benefit from treatment with the antibody or antigen-binding fragment of the another aspect of the invention.

In some embodiments, the present disclosure relates to a composition according to an aspect of the present disclosure, for use in the diagnosis of a cancer form selected from the group consisting of breast cancer, brain cancer, cancer of the CNS, lung cancer, prostate cancer, pancreatic cancer, skin cancer, lymphoma and sarcoma, or a metastasis of any one of said cancer forms. The cancer forms that can be detected and/or diagnosed by using a composition according to an aspect of the present disclosure and listed in detail in the section “Clinical conditions”.

It will be appreciated by the person skilled in the art that the process of detecting a biological marker for a disease, e.g. integrin alpha10 as in the present invention, or diagnosing a disease, e.g. by analyzing the expression of integrin alpha10 as in the present invention, can include comparing the tissue to be analyzed to a healthy, non-malignant or non-affected tissue. For example, a breast cancer sample can be compared to an unaffected area in the same tissue sample or to a healthy breast tissue sample. A brain cancer sample and/or a sample from a cancer of the CNS can be compared to an unaffected area in the same tissue sample or to a healthy brain or CNS tissue sample. A lung cancer sample can be compared to an unaffected area in the same tissue sample or to a healthy lung tissue sample. A prostate cancer sample can be compared to an unaffected area in the same tissue sample or to a healthy prostate tissue sample. A pancreatic cancer sample can be compared to an unaffected area in the same tissue sample or to a healthy pancreatic tissue sample. A sarcoma sample can be compared to an unaffected area in the same tissue sample or to a healthy connective tissue sample.

In the process of diagnosing a cancer, the person skilled in the art will appreciate the possible usefulness to compare the level of integrin alpha10 polypeptide or polynucleotide of a cancer cell to a reference cell.

In some embodiments, the diagnosed cancer comprises cells which display equal or higher levels of i) the integrin alpha10 antigen or ii) the polynucleotide transcript observed in healthy and/or benign tissue of the same type.

In some embodiments, the diagnosed cancer comprises cells which display equal or higher levels of i) the integrin alpha10 antigen or ii) the polynucleotide transcript observed in a less cancer type of the same tissue type, compared to the diagnosed cancer type.

In some embodiments, the diagnosed cancer comprises cells which display equal or higher expression levels of the integrin alpha10 antigen.

The person skilled in the art will appreciate that reference cell lines might be of use in a standardized diagnosis procedure due to the genotypic and phenotypic stability of these cell lines compared to primary cells. Reference cell lines might be established from healthy tissue or from cancer tissue, wherein the cancer might be a more or less aggressive cancer type, depending on its tendency to grow and metastasize. Established cell lines need to be immortalized to be able to be propagated in cell culture. A non-malignant cell line as used herein is understood as an established cell line which does not show signs of malignancy, and which is similar in its phenotype to healthy tissue cells.

In some embodiments, the reference cell line used during the diagnosis procedure is derived from healthy tissue, such as selected from the group consisting of a reference cell line derived from healthy breast tissue, a reference cell line derived from healthy prostate tissue, a reference cell line derived from healthy lung tissue, a reference cell line derived from healthy pancreas tissue and a reference cell line derived from healthy connective tissue.

In some embodiments, the reference cell line used during the diagnosis procedure of breast cancer is derived from healthy tissue.

Another aspect of the present disclosure relates to a method for detection and/or diagnosis of a cancer form in a subject, the method comprising the steps of:
a. providing a tissue suspected of comprising cancer cells of the subject;
b. analyzing in the tissue the presence of an antigen comprising an integrin alpha10 polypeptide or a fragment thereof;
c. determining the expression level of the integrin alpha10 antigen, and
d. comparing the expression level determined in c. with a control level, wherein said control level is the average expression level of the antigen observed in healthy and/or benign tissue of the same type; and
   wherein an expression level of the antigen higher than the control level is indicative of the presence of an cancer form in a sample, wherein said cancer form is selected from the group consisting of breast cancer, brain cancer, cancer of the CNS, lung cancer, prostate cancer, pancreatic cancer, skin cancer, lymphoma and sarcoma, or any cancer type listed herein under “Clinical conditions”, or a metastasis of any one of said cancer forms,
   thereby diagnosing a cancer form in a subject.

Another aspect of the present disclosure relates to a method for diagnosis of a cancer form in a subject, the method comprising the steps of:
a. providing a tissue suspected of comprising cancer cells of the subject;
b. analyzing in the tissue the presence of one or more cells having a cancer morphology;
c. analyzing in the sample the presence of an antigen comprising an integrin alpha10 polypeptide or a fragment thereof;
   optionally determining the expression level of the integrin alpha10 antigen, and wherein presence of one or more cells having a cancer morphology in combination with expression of integrin alpha10 antigen are indicative of the presence of an cancer form in a sample, wherein said cancer form is selected from the group consisting of breast cancer, brain cancer, cancer of the CNS, lung cancer, prostate cancer, pancreatic cancer, skin cancer, lymphoma and sarcoma, or any cancer type listed herein under “Clinical conditions”, or a metastasis of any one of said cancer forms, thereby diagnosing a cancer form in a subject.

In some embodiments, analyzing presence of an antigen comprising an integrin alpha10 polypeptide or a fragment thereof in step b. comprises contacting the tissue suspected of comprising cancer cells with a composition of the present disclosure. For example, in some embodiments, the tissue suspected of comprising cancer cells may be put in contact with a composition comprising or consisting of an antibody with specificity for an integrin alpha10 polypeptide.

In addition to analyzing in the sample the presence of an antigen comprising an integrin alpha10 polypeptide or a fragment thereof; and/or a polynucleotide transcript which encodes an integrin alpha10 polypeptide or a fragment or variant thereof, and determining whether the integrin alpha10 expression level is higher than a control level, the method for diagnosis of a cancer in a subject and/or the method for detecting a cancer cell in a subject may further comprise a step of morphologically characterizing the sample as comprising cancer cells belonging to a cancer, wherein said cancer form is selected from the group consisting of breast cancer, brain cancer, cancer of the CNS, lung cancer, prostate cancer, pancreatic cancer, skin cancer, lymphoma and sarcoma, or any cancer type listed herein under “Clinical conditions”, or a metastasis of any one of said cancer forms.

Another aspect, the present disclosure relates to a method for detecting a cancer cell in a subject, said method comprising the steps of:
a. providing a tissue suspected of comprising cancer cells of the subject;
b. analyzing in the tissue the presence of one or more cells having a cancer morphology,
c. analyzing in the tissue the presence of an antigen comprising an integrin alpha10 polypeptide or a fragment thereof;
d. optionally determining the expression level of the integrin alpha10 antigen,
   wherein presence of one or more cells having a cancer morphology in combination with expression of integrin alpha10 antigen are indicative of the presence of a cancer form in the subject, and wherein said cancer form is selected from the group consisting of breast cancer, brain cancer, cancer of the CNS, lung cancer, prostate cancer, pancreatic cancer, skin cancer, lymphoma and sarcoma, or any cancer type listed herein under “Clinical conditions”, or a metastasis of any one of said cancer forms.

Another aspect of the present disclosure relates to a method for determining a prognosis for a cancer form for a subject, the cancer form selected from the group consisting of breast cancer, brain cancer, cancer of the CNS, lung cancer, prostate cancer, pancreatic cancer, skin cancer, lymphoma and sarcoma, or a metastasis of any one of said cancer forms, the method comprising:
a. providing a cancer tumor tissue of the subject;
b. analyzing in the sample the presence of an antigen comprising an integrin alpha10 polypeptide or a fragment thereof;
c. determining the expression level of the integrin alpha10 antigen,
d. comparing the expression level determined in c. to a control level, wherein the control level is the average expression level of the integrin alpha10 antigen observed in healthy and/or benign tissue of the same tissue type as the sample;
e. determining an unfavourable prognosis of the cancer when the expression level of the antigen is higher than the control level.

Another aspect of the present disclosure relates to a method for determining a prognosis for a cancer for a subject, the method comprising:
a. providing a cancer tumor tissue of the subject;
b. analyzing in the tissue the presence of one or more cells having a cancer morphology;
c. analyzing in the sample the presence of an antigen comprising an integrin alpha10 polypeptide or a fragment thereof;
d. optionally determining the expression level of the integrin alpha10 antigen,
e. determining an unfavourable prognosis of the cancer form when one or more cells having a cancer morphology are present in the tissue in combination with expression of the integrin alpha10 antigen,
   wherein the cancer selected from the group consisting of breast cancer, brain cancer, cancer of the CNS, lung cancer, prostate cancer, pancreatic cancer, skin cancer, lymphoma and sarcoma, or a metastasis of any one of said cancer.

The method for determining a prognosis for a cancer for a subject may be applied to any one of the cancer forms described herein, see for example the section “Clinical conditions”.

In some embodiments, the present disclosure relates to a method for determining a prognosis for a cancer form for a subject, wherein the prognosis is overall survival rate or recurrence free survival rate.

In some embodiments, the methods disclosed herein target an antigen comprising an integrin alpha10 polypeptide or a fragment thereof that is expressed on the surface of the cells.

The methods of the present disclosure may be conducted in vivo or in vitro.

In some embodiments, the method for detecting a cancer cell in a subject as disclosed herein is conducted in vivo. In some embodiments, the method for diagnosis of a cancer in a subject as disclosed herein is conducted in vitro, and the tissue is a tissue sample obtained from the subject.

In some embodiments, the method for diagnosis of a cancer in a subject as disclosed herein is conducted in vivo. In some embodiments, the method for diagnosis of a cancer in a subject as disclosed herein is conducted in vitro, and the tissue is a tissue sample obtained from the subject.

In some embodiments, the method for determining a prognosis for a cancer in a subject as disclosed herein is conducted in vivo. In some embodiments, the method for determining a prognosis for a cancer in a subject as disclosed herein is conducted in vitro, and the tissue is a tissue sample obtained from the subject.

In some embodiments, the step of analyzing in the sample the presence of an antigen comprising an integrin alpha10 polypeptide or a fragment thereof comprises imaging the tissue and/or tissue sample.

In some embodiments, the step of determining the expression level of the integrin alpha10 antigen comprises imaging the tissue and/or tissue sample. In some embodiments, the step of determining the expression level of the integrin alpha10 antigen comprises imaging a tissue in vivo, such as during open-brain surgery.

Imaging may for example be conducted by administering to the subject and/or to the tissue sample a labelled moiety that is capable of binding to an antigen comprising an integrin alpha10 polypeptide or a fragment thereof. For example, imaging may be conducted by administering to the subject and/or to the tissue sample a labelled integrin alpha10 antibody as defined herein.

In some embodiments, the integrin alpha10 antibody administered as a part of an antibody-drug conjugation (ADC). In an ADC, the antibody is linked to a moiety e.g. a disease modifying drug or toxin. Upon specifically binding to integrin alpha10 polypeptide, the ADC is internalized into the cell, and thereby the moiety is delivered into the cell.

In some embodiments, the antibody comprised in the composition for use in the diagnosis and/or treatment of a cancer form of the present disclosure is an antibody fragment.

In some embodiments, the antibody specifically binding to an integrin alpha10 polypeptide or a fragment thereof according to the present disclosure may be conjugated to a moiety, such as an additional moiety. The conjugation may improve and facilitate both treatment and diagnosis of a cancer form.

### EXAMPLES

### Example 1: Antibody Production/Humanization

### 1.1 Design of Composite Human Antibody Variable Regions

Aim: To generate human antibody sequence segments to create humanised antibody variants.

Material and Methods: Structural models of the mouse antibody mAb365 V regions were produced. Based on structure analysis, humanized variants sequences, likely to be essential for the binding properties of the antibody, were selected and analysed in silico. Human sequence segments were identified for CDR regions and for regions outside of the CDRs. Selected sequence segments were assembled to generate complete humanized V region sequences that were devoid of, or reduced in, significant T cell epitopes to avoid immunogenicity.

Results: The design resulted in six heavy chain (VH1 to VH6) and five light chain (VK1 to VK5) sequences that were used for gene synthesis and expression in mammalian cells (Table 1). The VH4 variant, being part of the CDR4 region was excluded due to the risk that changes in this region may affect the CDR confirmation. Five VH were combined with 5 VK generating 25 different humanized variants.

Conclusion: Twenty-five humanized variants and one chimeric variant were generated.

TABLE 1
Summary table of the 25 humanized antibody variants including
a chimeric (TAR-Ab0). The VH4 was avoided to make variants
due to the risk of changing CDR conformation.

| | VH0 | VH1 | VH2 | VH3 | VH4 | VH5 | VH6 |
|---|---|---|---|---|---|---|---|
| VK0 | ✓ | | | | | | |
| VK1 | | ✓ | ✓ | ✓ | x | ✓ | ✓ |
| VK2 | | ✓ | ✓ | ✓ | x | ✓ | ✓ |
| VK3 | | ✓ | ✓ | ✓ | x | ✓ | ✓ |
| VK4 | | ✓ | ✓ | ✓ | x | ✓ | ✓ |
| VK5 | | ✓ | ✓ | ✓ | x | ✓ | ✓ |

### 1.2 Construction and Transient Expression of Chimeric IgG1 and Humanized IgG1 Variants

Aim: To generate small scale batches of antibodies for lead candidate selection.

Material and Methods: The chimeric Tar-Ab0 sequences and the humanized variants were used to express IgG1 antibodies in CHO cells. Culture supernatants were harvested on day 6 post-transfection and antibody concentrations were measured.

Results and Conclusion: 17 different variants had improved yields as compared to the chimeric antibody (TAR-Ab0) (data not shown). TAR-Ab23 had 3.9 fold better yield than the chimeric antibody.

### 1.3 Selection of Five Lead Candidates

Aim: To select five lead candidates from 25 humanized variants based on binding ability.

Material and methods: The mouse myoblast cell line C2C12 overexpressing either human integrin alpha10beta1 vector (C2C12alpha10) or human integrin alpha11beta1 (C2C12alpha11) were used to study antibody binding and specificity. The C2C12 cells were incubated (100 000 cells/sample) with the integrin alpha10 antibodies at 1 μg/ml for 30 min followed by a secondary antibody for 30 min and the binding was then analysed by flow cytometry.

Results: The results showed that the chimeric format TAR-Ab0 as well as humanized variants TAR-Ab3, TAR-Ab8, TAR-Ab9, TAR-Ab13, TAR-Ab14 and TAR-Ab23, and have the best binding affinity to C2C12alpha10 cells (data not shown). None of the antibodies show binding to the control C2C12alpha11 cells (data shown). Additionally, risk analysis data from the sequence design process suggest that TAR-Ab3 has the highest risk scores (data not shown). Therefore, variants TAR-Ab8, TAR-Ab9, TAR-Ab13, TAR-Ab14, and TAR-Ab23 were selected for further functional studies.

Conclusion: Five humanized antibody lead candidates with specific binding to integrin alpha10beta1 were selected: TAR-Ab8, TAR-Ab9, TAR-Ab13, TAR-Ab14, and TAR-23.

### 1.4 Functionality Properties of the Five Antibody Lead Candidates

Aim: To investigate the function blocking effect of the five lead candidates on proliferation, adhesion and migration of integrin alpha10beta1-expressing cells.

Materials and Methods: The mouse myoblast cell line C2C12 overexpressing the integrin alpha10beta1 (C2C12alpha10) was used in the function blocking studies.

### Cell Proliferation

C2C12alpha10 cells, mixed with integrin alpha10 antibodies at final concentrations of 5 μg/ml, were seeded in 96-well plates (10 000 cells per well) precoated with collagen type I. After 24 hours of treatment with antibodies, bromo deoxyuridine (BrdU) was added for 2 hours and proliferation was measured using the Cell Proliferation ELISA BrdU kit (Roche Diagnostics GmbH) according to the manufacturer's instructions.

### Cell Adhesion

Culture plates (48-well) were coated over night with collagen type IV (10 μg/ml) or bovine serum albumin (BSA) as a control. Prior to the experiments the plates were incubated with 0.25% BSA at 37° C. for 30 min to block non-specific binding. BT549 cells were pre-incubated for 20 minutes in the presence or in the absence of integrin alpha10 antibodies (5 μg/ml) and then allowed to attach to the coated dishes for 60 min at 37° C. Non-adherent cells were washed away and adherent cells were fixed with ethanol and stained with 0.09% crystal violet. The absorbed dye was then extracted by 10% acetic acid and quantified by optical density (OD) at 590 nm.

### 3D Collagen Gel Migration

Collagen gel solution was made by mixing collagen type I (6 mg/ml C2124, Sigma Aldrich) with culture medium to a final concentration of 1.2 mg/ml. The collagen gel solution (100 μl volume) was added to a 96-well plate and incubated for 15 min at 37° C. Spheroids of C2C12alpha10 cells were then placed into the collagen solution with a pipette and the collagen-spheroid solution was allowed to polymerize for 60-90 min at 37° C. After polymerization, 100 μl medium containing 10 μg/ml of integrin alpha10 antibodies or control antibodies were added to the wells to cause the collagen gel to float in the antibody solutions. Migration of C2C12alpha10 cell from the spheroids into the collagen gels was monitored using an inverted lighted microscope and photographed at different time points for quantification.

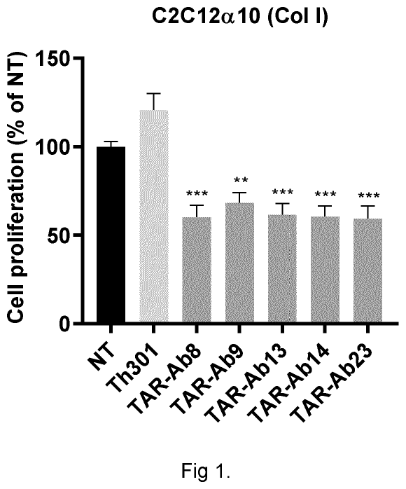

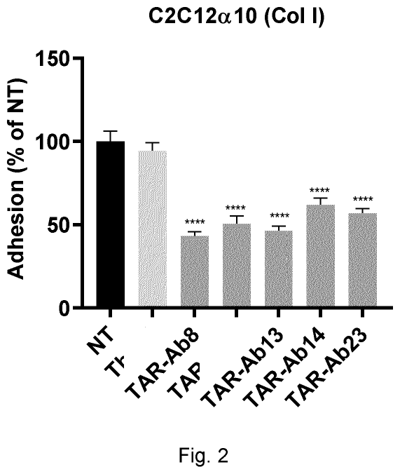

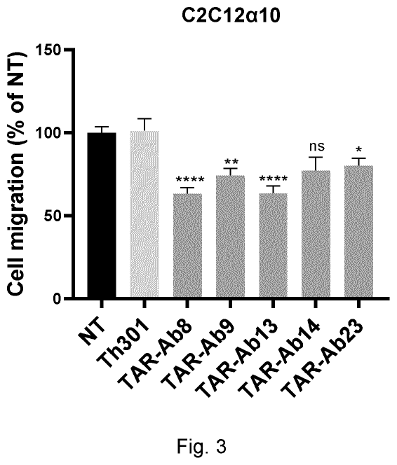

Results: The results showed that all the 5 antibody lead candidates significantly reduced the cell proliferation (FIG. 1) and adhesion (FIG. 2) of C2C12alpha10 cells while the isotype control antibodies did not show the inhibitory effect on the C2C12alpha10 cells (FIG. 1-2). In 3D migration assay, all the humanized variants as well as mAb365 except TAR-Ab14 gave significant reduction on cell migration (FIG. 3).

Conclusion: All five humanized antibody variants show strong function blocking properties in proliferation, adhesion and migration assays.

### Example 2: Thermal Stability Analysis

Aim: To rank the top five humanized antibody variants based on stability.

Material and Methods: Thermal stability analysis of the humanized antibody variants, was performed using a Uncle™ biostability platform and software. Samples for each variant were formulated in PBS and Sypro Orange at a final concentration of 0.5 mg/ml. Samples were subjected to a thermal ramp from 25-95° C., with a ramp rate of 0.3° C./minute and excitation at 473 nm. Monitoring of static light scattering (SLS) at 473 nm allowed the detection of protein aggregation, and T_{agg} (onset of aggregation) was calculated from the resulting SLS profiles.

Results: All humanized antibody variant showed_good pharmaceutical properties, including high thermostability and low aggregation propensity which will facilitate manufacturing and storage and suggests long serum half-life. All humanized variants showed higher calculated melting temperature (Tm) than the chimeric antibody (Tar-Ab0). The most thermally stable variants were TAR-Ab23, TAR-Ab13, TAR-Ab8 (Table 4). The thermal stability of TAR-Ab23 was 17.4° C. higher than for the chimeric variant.

Conclusion: The thermal stability was higher for all the five selected humanized antibody variants compared to the chimeric antibody variant. This which supports therapeutic use of the humanized antibodies.

TABLE 4
Summary of thermal stability values for the
five lead variants and chimeric antibody.

| Variant | AvTₘ (° C.) |
|---|---|
| TAR-Ab0 | 50.0 |
| TAR-Ab8 | 65.3 |
| TAR-Ab9 | 55.3 |
| TAR-Ab13 | 67.3 |
| TAR-Ab14 | 60.6 |
| TAR-Ab23 | 67.4 |

### Example 3: Isoelectric Point

Aim: To determine the isoelectric points (PI) of the humanized antibody variants in comparison with the chimeric antibody.

Material and Methods: Isoelectric point (pI) of an antibody is the pH at which the antibody has no net electrical charge, and its value depends on the charged amino acids the antibody contains. The isoelectric points of the antibody variants were calculated based on the DNA/Protein sequence analysis software.

Results: The analysis shows that TAR-Ab23 has pI of 8.121 while the chimeric antibody Tar-Ab0 has pI of 7.83. This is an advantage since a higher pI is associated with proportionally higher drug exposure in the tumor and thus improves the potential for inducing a positive clinical effect.

Conclusion: TAR-Ab23 has a higher isoelectric point than chimeric antibody which support higher therapeutic potential. Therapeutic antibodies often have a pI that is higher than the pH in the biophase. Tumors are usually acidic due to the formation of lactic acid. As a consequence, antibodies with elevated PI generally have an improved tumor to blood ratio compared to antibodies with a lower PI. An elevated PI is therefore considered an advantage for a therapeutic antibody for cancer therapy.

### Example 4: Binding Affinity of TAR-Ab23 for Integrin Alpha10beta1

Aim: To determine integrin alpha10 binding specificity and affinity of TAR-Ab23 in comparison to mAb365.

Material and Methods: Integrin alpha10beta1 overexpressing cells (C2C12alpha10), human patient derived glioblastoma cells (U3046MG) and triple negative breast cancer cells (BT549) were used. The cells were incubated with the humanised (TAR-Ab23) or the mouse (mAb365) antibodies at different concentrations (0.1, 1, 10, 100, 1000 nM) for 30 min at 4° C., and the binding was then analysed by flow cytometry.

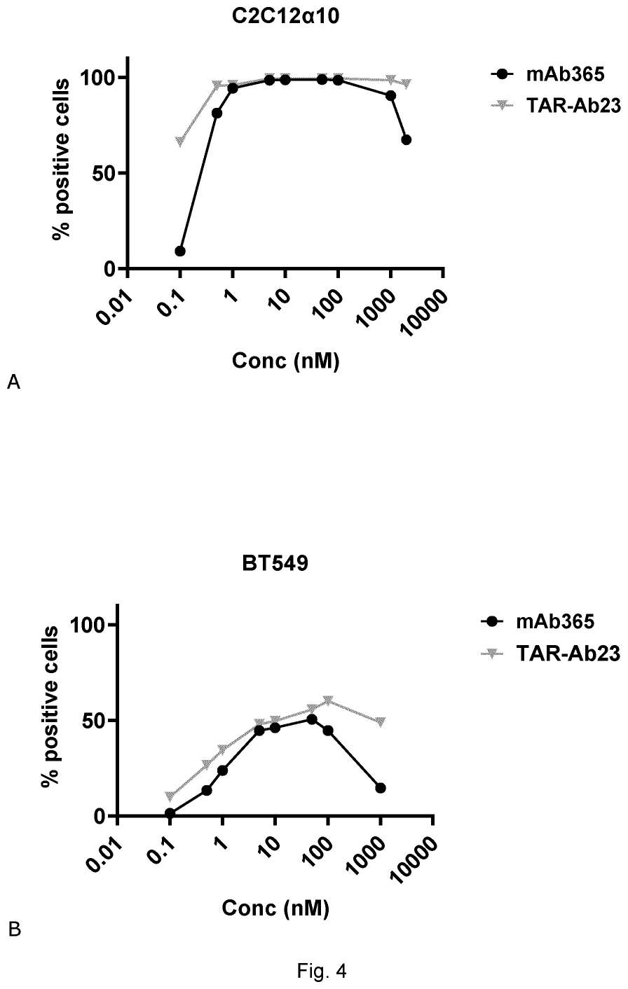

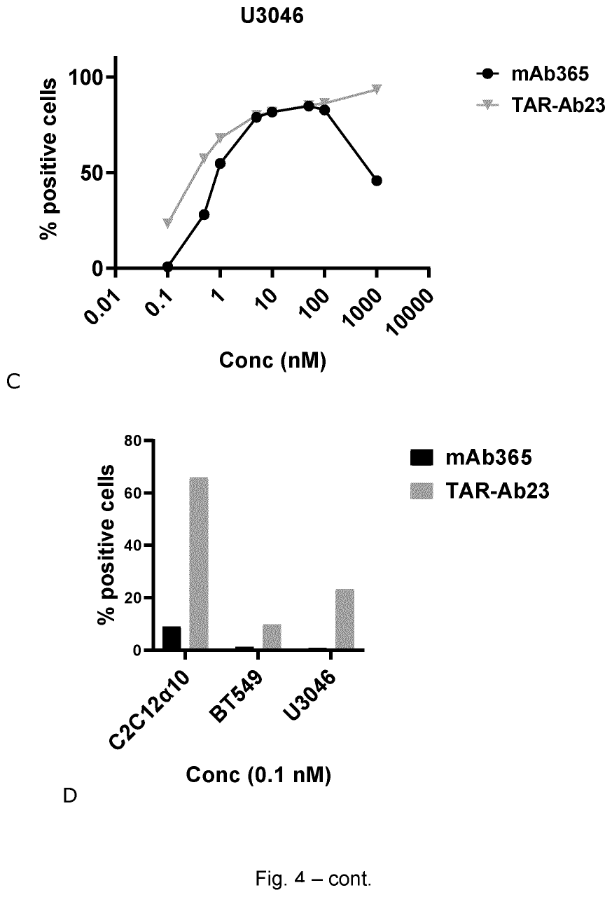

Results: Both TAR-Ab23 and mAb365 have good binding affinity for all three cell lines (C2C12alpha10, U3046MG, and BT549) (FIG. 4A-C). However, TAR-Ab23 demonstrated higher binding to the cells compared mAb365 especially at lower concentrations e.g. 0.1 nM (FIG. 4D). Furthermore, the binding capacity of mouse antibody mAb365 decreased at the higher concentrations (above 100 nM), likely due to aggregation, while the binding of the humanized antibody TAR-Ab23 remained high (FIG. 4).

### Conclusion:

These findings showed that TAR-Ab23 has higher binding affinity than mAb365 and that TAR-Ab23 is more stable than mAb365 at high concentrations (>100 nM).

### Example 5: Specific Binding of TAR-Ab23 to TNBC Tissues

Aim: To demonstrate specific binding of TAR-Ab23 to integrin alpha10beta1 on cancer cells in primary TNBC tissues.

Material and Methods: Triple negative breast cancer human tissues consisting of adenocarcinoma of breast, ductal, metastatic case was investigated for immunohistochemical staining of integrin alpha10 antibodies. Sections were stained with the humanized TAR-Ab23 antibody, the mouse mAb365 antibody and the isotype control antibodies human IgG1 and mouse IgG2a (all antibodies at 3 μg/ml).

Results: The TAR-Ab23 antibody showed specific and strong staining of cancer cells in TNBC tissue. In contrast, mammary ducts in the adjacent normal tissues exhibited low or no TAR-Ab23 staining. The mAb365 antibody showed non-specific nuclear staining in the TNBC in addition to expected integrin alpha10 staining of the TNBC cells. The isotype control antibodies did not reveal any detectable staining.

Conclusion: TAR-Ab23 can be used to detect and target integrin alpha10beta1 in tissues due to its high and specific binding to the integrin subunit alpha10. The antibody mAb365, on the other, hand showed non-specific nuclear staining of cells both in the tumor and in the surrounding tissue. This supports the use of TAR-Ab23 both as a companion diagnostic tool and as therapeutic drug in treatment of TNBC and other cancer forms.

### Example 6: Binding Competition Assay Between the Integrin Alpha10 Antibodies Humanized TAR-Ab23 and Mouse mAb365

Aim: To investigate the affinity of the antibodies TAR-Ab23 and mAb365 to integrin alpha10 in a quantitative competition assay using flow cytometry.

Material and Methods: A binding competition assay of the antibodies TAR-Ab23 and mAb365 was performed with the triple negative breast cancer cell line BT549, the glioblastoma cell line U3054MG and with C2C12alpha10 cells. The antibodies were added simultaneously to the cells at the concentration of 0.1 nM. After incubation for 30 min, secondary antibodies were added for another 30 min. Donkey anti-human Alexa 488 was used as a secondary antibody to the human antibody TAR-Ab23. Donkey anti-mouse 20 Alexa 647 was used as a secondary antibody to the mouse antibody mAb365. The binding of the antibodies was analysed by flow cytometry.

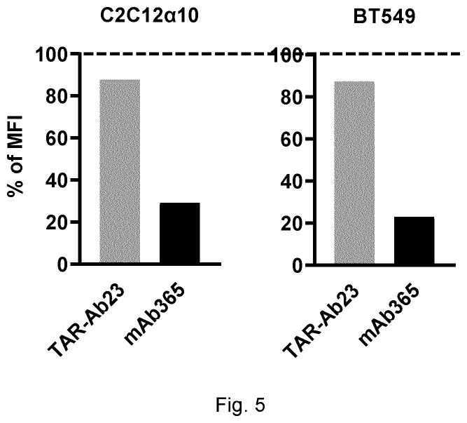

Results: The competition assay showed that TAR-Ab23 bound to the majority of the C2C12alpha10 cells (87.6%) and BT549 cells (87.3%) while mAb365 bound only 29.1% of C2C12alpha10 cells and 22.9% of BT549 cells (FIG. 5). Additionally, TAR-Ab23 did not completely compete out the binding of mAb365 suggesting a small overlap of binding epitopes between two antibodies.

Conclusion: TAR-Ab23 has more than 3-fold higher binding affinity than mAb365 to integrin alpha10 and also binds faster to the integrin alpha10 antigen compared to mAb365.

### Example 7: Function-Blocking Properties of the Antibody TAR-Ab23 In Vitro Inhibition on Adhesion, Proliferation and Migration after Blocking Integrin Alpha10beta1 with TAR-Ab23

### 7.1 Adhesion

Aim: To investigate the effect of TAR-Ab23 on adhesion of TNBC cells in comparison with mAb365.

Material and Methods: Culture plates (48-well) were coated over night with collagen type IV (10 μg/ml) or bovine serum albumin (BSA) as a control. Prior to the experiments the plates were incubated with 0.25% BSA at 37° C. for 30 min to block non-specific binding. BT549 cells were pre-incubated for 20 minutes in the presence or in the absence of integrin alpha10 antibodies (5 μg/ml) and then allowed to attach to the coated dishes for 60 min at 37° C. Non-adherent cells were washed away and adherent cells were fixed with ethanol, stained with 0.09% crystal violet. The absorbed dye was then extracted by 10% acetic acid and quantified by optical density (OD) at 590 nm.

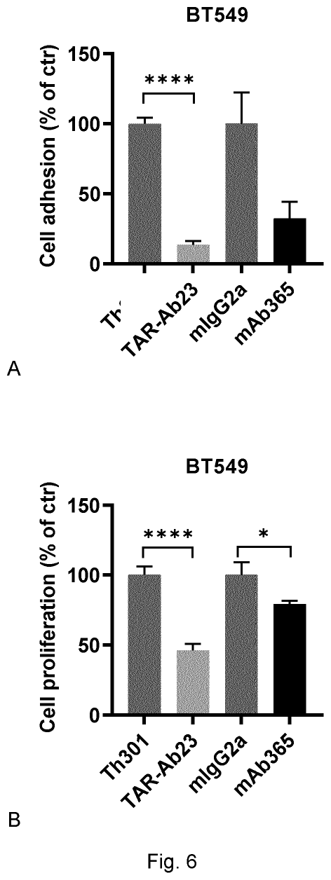

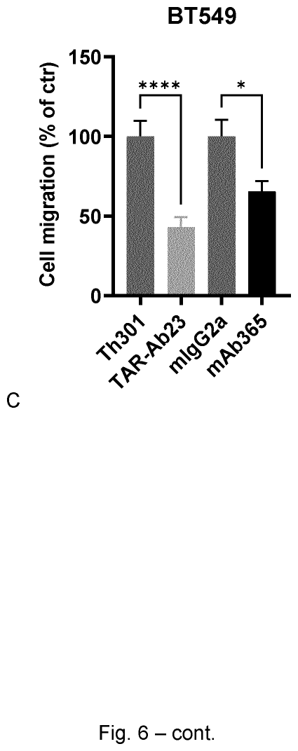

Results: TAR-Ab23 significantly inhibited adhesion of BT549 cells to collagen IV-coated dishes (86% reduction). The effect of TAR-Ab23 was more pronounced compared to mAb365 (70% reduction) (FIG. 6A).

Conclusion: TAR-Ab23 is more efficient than mAb365 in inhibiting integrin alpha10-dependent adhesion to the extracellular matrix component collagen type IV.

### 7.2 Proliferation

Aim: To investigate the effect of the TAR-Ab23 on proliferation of TNBC cells in comparison with mAb365.

Material and Methods: BT549 cells were seeded in 96-well plates coated with collagen type IV and treated with integrin alpha10 or control antibodies at a final concentration of 5 μg/ml. After 48 hours of treatment with antibodies, bromo deoxyuridine (BrdU) was added for 3 hours and proliferation (BrdU incorporation in DNA) was measured using the Cell Proliferation ELISA BrdU kit (Roche Diagnostics GmbH).

Results: Treatment of the BT549 cells with the integrin alpha10 antibodies TAR-Ab23 and mAb365 decreased cell proliferation compared to treatment with the respective control antibodies (Th301 and mIgG2a; FIG. 6B). However, TAR-Ab23 inhibited cell proliferation more efficiently than mAb365.

Conclusion: TAR-Ab23 has inhibiting effect on TNBC cells and is more efficient compared to mAb365.

### 7.3 3D Migration

Aim: To investigate the effect of TAR-Ab23 on 3D migration of TNBC cells in collagen gels in comparison with mAb365.

Material and Methods: Collagen gel solution was made by mixing collagen type I (6 mg/ml C2124, Sigma Aldrich) with culture medium to a final concentration of 1.2 mg/ml. The collagen gel solution (100 μl) was added to a 96-well plate and incubated for 15 min at 37° C. Spheroids of BT549 cells were then placed into the collagen solution with a pipette and the collagen-spheroid solution was allowed to polymerize for 60-90 min at 37° C. After polymerization, 100 μl medium containing 10 μg/ml of the integrin alpha10 antibodies or control antibodies were added to the wells to cause the collagen gel to float in the antibody solutions. Migration of BT549 from the spheroids into the collagen gels was monitored and photographed at different time points using an inverted lighted microscope for quantification.

Results: Both antibodies TAR-Ab23 and mAb365 inhibited migration of BT549 cells while control antibodies (Th301 or mIgG2a) had no effect (FIG. 6C). However, the inhibitory effect of TAR-Ab23 was more pronounced than mouse, mAb365. TAR-Ab23 reduced migration of BT549 cells in the collagen gel by 57% while mAb365 reduced migration by 45% compared to the control antibodies (FIG. 6C).

Conclusion: The results suggest that the TAR-Ab23 inhibits integrin alpha10-mediated cell migration more effectively than mAb365.

The function blocking properties of integrin alpha10 antibody in vitro demonstrate that TAR-Ab23 can strongly block anchorage dependent cell functions such as adhesion, proliferation and migration.

### Example 8: Function-Blocking Properties of the Antibody TAR-Ab23 In Vivo

Aim: To investigate treatment effect of TAR-Ab23 on metastasis of the triple negative breast cancer cell line MDA-MB-231.

Material and Methods: MDA-MB-231 cells, labelled with Luciferase/GFP (2×10⁶ cells) were injected intravenously through tail vein into four weeks old NMRI-nu immunodeficient mice (Janvier, France). One day after inoculation, D-luciferin substrate was injected subcutaneously and the mice were the subjected to live imaging with IVIS-CT to examine tumor progression. Based on the bioluminescence images (Total flux read out (photon/sec)), mice were randomly categorized into three different treatment groups and the antibodies (5 mg/kg) were injected intraperitoneally. Mice were imaged by IVIS-CT once a week to monitor metastasis. Total body weight was measured bi-weekly.

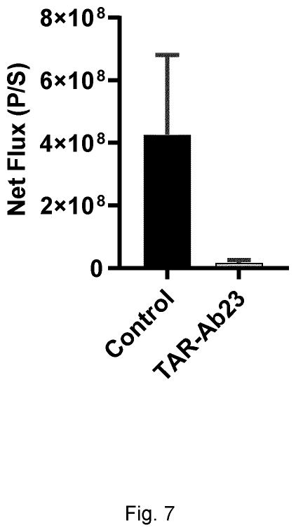

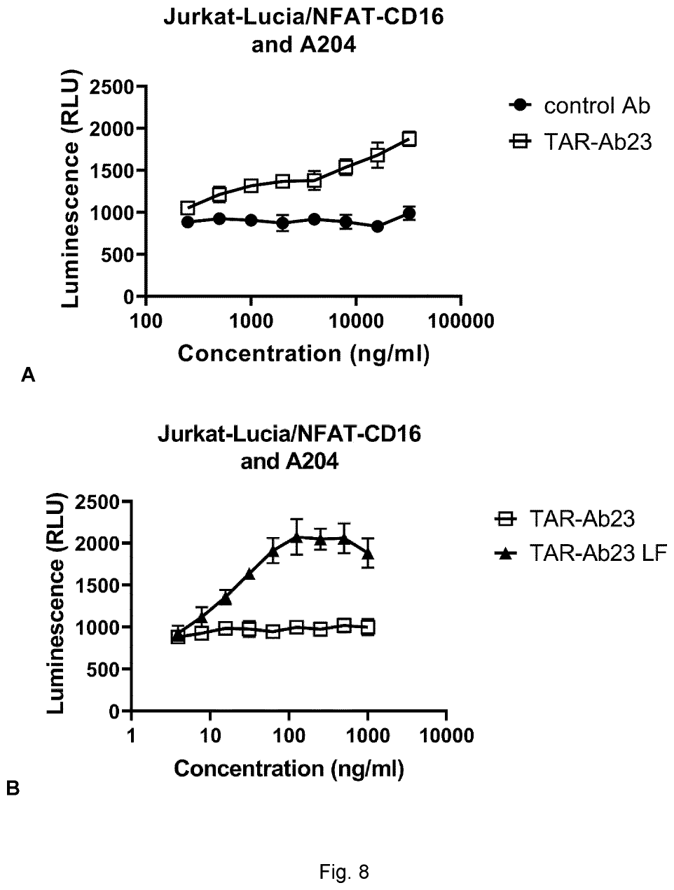

Results: Imaging analysis demonstrated that TAR-Ab0 treatment inhibited spontaneous metastasis in mice, including lung and liver metastasis, after intravenous injection of MDA-MB-231 (FIG. 7).

Conclusion: The results show that TAR-Ab23 has a direct antimetastatic effect on TNBC in the xenograft model. This indicates that the cell surface molecule integrin alpha10beta1 is important for metastasis of TNBC cells and may represent a promising target for therapeutic cancer intervention.

### Example 9: The Activity of a Therapeutic Antibodies is Mediated in Part by Antibody-Dependent Cell-Mediated Cytotoxicity (ADCC)

Aim: To investigate if TAR-Ab23 possess the ability to activate an ADCC response.

An optimized variant of TAR-Ab23 with potential for stronger ADCC has been generated by decreasing fucosylation. The TAR-Ab23 low fucose versus TAR-Ab23 was also used to quantify the ADCC activity in the gene reporter assay.

Material and Methods: Jurkat Lucia NFAT-CD16 effector cells were passaged two days prior to the ADCC assay. On the day prior to assay, the A204 target cells were seeded in complete medium in flat-bottom 96 well plate (SPL) based on the effector/target cell ratio according to plate layout. On the day of ADCC assay, the cell culture medium from the 96 well plate with attached target cells was removed and 90 ul fresh test medium (exp #1-3: RPMI-1640+10% heat-inactivated FBS, exp #4-6: RPMI-1640+2% heat-inactivated low IgG FBS) was added. To the target cells, 20 μl of the antibody dilution series was added per well and incubated for 1 hour. After incubation 90 μl of Jurkat-Lucia NFAT-CD16 effector cells were added according to effector/target cell ratio (2:1). Plate was incubated for 24 hours. 20 μl of cell supernatant was transferred into a 96-well white (opaque) plate and 50 μl of QUANTI-Luc was added per well and subjected immediately to measurement in luminometer.

Results: The ADCC activity of TAR-Ab23 (A) and TAR-Ab23 low fucose (B) were determined by using Jurkat Lucia/NFAT-CD16 cells. The TAR-Ab23 produced a dose-dependent signal curve compared to the control antibody by showing ADCC activity in A204 rhabdoid cell line. The ADCC activity enhanced significantly by decreasing fucose content via afucosylation. TAR-mAb23 low fucose resulted in a dose-dependent ADCC response compared to parental antibody at much lower concentration.

Conclusion: Our data reveal that TAR-Ab23 induces ADCC. Furthermore, TAR-Ab23 low fucose variant showed to have greater ADCC activity.

### Example 10: Sequences of the Antibody

SEQ ID NO: 1:
RASKSVSTSGFSHMH

Variable light chain complementarity-determining region 1 (CDR-L1), constant between the 5 light chain humanized variants.

SEQ ID NO: 2:
LASNLES

Variable light chain complementarity-determining region 2 (CDR-L2) of VK1, VK2, and VK3.

SEQ ID NO: 3:
LGSNLES

Variable light chain complementarity-determining region 2 (CDR-L2) of VK4.

SEQ ID NO: 4:
GASNLES

Variable light chain complementarity-determining region 2 (CDR-L2) of VK5.

SEQ ID NO: 5:
QHSRELPRT

Variable light chain complementarity-determining region 3 (CDR-L3), constant between the 5 light chain humanized variants.

SEQ ID NO: 6:
DYNMD

Variable heavy chain complementarity-determining region 1 (CDR-H1) of VH1, VH2 and VH3.

SEQ ID NO: 7:
DYNMN

Variable heavy chain complementarity-determining region 1 (CDR-H1) of VH5

SEQ ID NO: 8:
DYNMH

Variable heavy chain complementarity-determining region 1 (CDR-H1) of VH6.

SEQ ID NO: 9:
DINPNTGGTIYNQKFKG

Variable heavy chain complementarity-determining region 2 (CDR-H2), constant between the 5 heavy chain humanized variants.

SEQ ID NO: 10:
REDWYYFDF

Variable heavy chain complementarity-determining region 3 (CDR-H3), constant between the 5 heavy chain humanized variants.

(Variable light chain-VK1)
SEQ ID NO: 11
DIVLTQSPATLALSPGERATLSCRASKSVSTSGFSHMHWYQQKPGQPPK
LLIYLASNLESGVPSRFSFSGSGTDFTLNIHPVQEEDAATYYCQHSREL
PRTFGGGTKVEIK
(Variable light chain-VK2)
SEQ ID NO: 12
DIVLTQSPATLSLSPGERATLSCRASKSVSTSGFSHMHWYQQKPGQPPK
LLIYLASNLESGVPSRFSFSGSGTDFTLTISSLQPEDVATYYCQHSREL
PRTFGGGTKVEIK
(Variable light chain-VK3)
SEQ ID NO: 13
DIVLTQSPATLSLSPGERATLSCRASKSVSTSGFSHMHWYQQKPGQPPK
LLIYLASNLESGVPSRFSGSGSGTDFTLTISSLQPEDVATYYCQHSREL
PRTFGGGTKVEIK
(Variable light chain-VK4)
SEQ ID NO: 14
DIVLTQSPATLSLSPGERATLSCRASKSVSTSGFSHMHWYQQKPGQPPK
LLIYLGSNLESGVPSRFSFSGSGTDFTLTISSLQPEDVATYYCQHSREL
PRTFGGGTKVEIK
(Variable light chain-VK5)
SEQ ID NO: 15
DIVLTQSPATLSLSPGERATLSCRASKSVSTSGFSHMHWYQQKPGQPPK
LLIYGASNLESGVPSRFSFSGSGTDFTLTISSLQPEDVATYYCQHSREL
PRTFGGGTKVEIK
(Variable heavy chain-VH1)
SEQ ID NO: 16
EVQLVQSGPELKKPGASVKVPCKASGYTFTDYNMDWVRQPHGKGLEWIG
DINPNTGGTIYNQKFKGRATITVDKSTSTAYMELSSLRSEDTSVYYCAR
REDWYYFDFWGQGTTVTVSS
(Variable heavy chain-VH2)
SEQ ID NO: 17
EVQLVQSGAEVKKPGASVKVSCKASGYTFTDYNMDWVRQPPGKGLEWIG
DINPNTGGTIYNQKFKGRATITVDKSTSTAYMELSSLRSEDTSVYYCAR
REDWYYFDFWGQGTTVTVSS
(Variable heavy chain-VH3)
SEQ ID NO: 18
EVQLVQSGAEVKKPGASVKVSCKASGYTFTDYNMDWVRQPPGKGLEWIG
DINPNTGGTIYNQKFKGRATITVDKSTSTAYMELSSLRSEDTAVYYCAR
REDWYYFDFWGQGTTVTVSS
(Variable heavy chain-VH5)
SEQ ID NO: 19
EVQLVQSGAEVKKPGASVKVSCKASGYTFTDYNMNWVRQPPGKGLEWIG
DINPNTGGTIYNQKFKGRATITVDKSTSTAYMELSSLRSEDTAVYYCAR
REDWYYFDFWGQGTTVTVSS
(Variable heavy chain-VH6)
SEQ ID NO: 20
EVQLVQSGAEVKKPGASVKVSCKASGYTFTDYNMHWVRQPPGKGLEWIG
DINPNTGGTIYNQKFKGRATITVDKSTSTAYMELSSLRSEDTAVYYCAR
REDWYYFDFWGQGTTVTVSS

### REFERENCES

Bengtsson T, Aszodi A, Nicolae C, Hunziker E B, Lundgren-Akerlund E, Fassler R. Loss 15 of alpha10beta1 integrin expression leads to moderate dysfunction of growth plate chondrocytes. J Cell Sci 2005; 118:929-36.
Bondreau, N. J. and Jones, P. L. Extracellular matrix and integrin signalling: the shape of things to come. Biochem. J. 1999; 339:481-488.
Campbell I D, Humphries M J. Integrin structure, activation, and interactions. Cold Spring Harb Perspect Biol. 2011; 3(3).
Bengtsson T, Camper L, Schneller M, Lundgren-Akerlund E. Characterization of the mouse integrin subunit alpha10 gene and comparison with its human homologue. Genomic structure, chromosomal localization and identification of splice variants. Matrix Biol. 2001; 20(8):565-76.
Camper L, Holmvall K, Wängnerud C, Aszódi A, Lundgren-Akerlund E. Distribution of the collagen-binding integrin alpha10beta1 during mouse development. Cell Tissue Res. 2001; 306(1):107-16.
Chmielarska Masoumi K, Huang X, Sime W, Mirkov A, Munksgaard Thorén M, Massoumi R, Lundgren-Åkerlund E. Integrin α10-Antibodies Reduce Glioblastoma Tumor Growth and Cell Migration. Cancers (Basel). 2021; 13(5):1184.
Foote J, Winter G. Antibody framework residues affecting the conformation of the hypervariable loops. J Mol Biol. 1992 Mar. 20; 224(2):487-99.
Frith J E, Mills R J, Hudson J E, Cooper-White J J. Tailored integrin-extracellular matrix interactions to direct human mesenchymal stem cell differentiation. Stem Cells Dev. 35 2012; 21(13):2442-56.
Gullberg D E, Lundgren-Akerlund E. Collagen-binding I domain integrins—what do they do? Prog Histochem Cytochem. 2002; 37(1):3-54.
Heino J. The collagen receptor integrins have distinct ligand recognition and signalling functions. Matrix Biology. 2000; 19:319-323.
Hering T M. Regulation of chondrocyte gene expression. Frontiers in Bioscience. 1999; 4:743-761.
Hohenester E. Signalling complexes at the cell-matrix interface. Curr Opin Struct Biol. 2014; 29:10-6.
Jones P T, Dear P H, Foote J, Neuberger M S, Winter G. Replacing the complementarity-determining regions in a human antibody with those from a mouse. Nature. 1986; 4; 321(6069):522-5.
Kyöstilä K, Lappalainen A K, Lohi H. Canine chondrodysplasia caused by a truncating mutation in collagen-binding integrin alpha subunit 10. PLoS One. 2013; 8(9):e75621.
Lundgren-Åkerlund E, and Aszòdi A. Integrin alpha10beta1: a collagen receptor critical in skeletal development. Adv. Exp. Med. Biol. 2014; 819:61-71.
Munksgaard Thorén M, Chmielarska Masoumi K, Krona C, Huang X, Kundu S, Schmidt L, Forsberg-Nilsson K, Floyd Keep M, Englund E, Nelander S, Holmqvist B, Lundgren-Åkerlund E. Integrin α10, a Novel Therapeutic Target in Glioblastoma, Regulates Cell Migration, Proliferation, and Survival. Cancers (Basel). 2019; 25; 11(4):587.
Quenn C, Schneider W P, Selick H E, Payne P W, Landolfi N, Duncan J F, Avdalovic N M, Levitt M, Junghans R P, and Waldmann T. A humanized antibody that binds to the interleukin 2 receptor. Proc. Nati. Acad. Sci. 1989; 86, 10029-10033.
Riechmann L, Clark M, Waldmann H, Winter G. Reshaping human antibodies for therapy. Nature. 1988; 24; 332(6162):323-7.
Sanada H, Kobayashi K, Oyama K, Maru T, Nakanishi T, Umetsu M, Asano R, Kumagai I. Affinity maturation of humanized anti-epidermal growth factor receptor antibody using a modified phage-based open sandwich selection method. Sci Rep. 2018; 8(1):5414.
Sielska M, Przanowski P, Pasierbińska M, Wojnicki K, Poleszak K, Wojtas B, Grzeganek D, Ellert-Miklaszewska A, Ku MCh, Kettenmann H, Kaminska B. Tumour-derived CSF2/granulocyte macrophage colony stimulating factor controls myeloid cell accumulation and progression of gliomas. Br J Cancer. 2020; 123(3):438-448.
Varas L, Ohlsson L B, Honeth G, Olsson A, Bengtsson T, Wiberg C et al. Alpha10 integrin expression is up-regulated on fibroblast growth factor-2-treated mesenchymal stem cells with improved chondrogenic differentiation potential. Stem Cells Dev 2007; 16:965-978.
Uvebrant K, Reimer Rasmusson L, Talts J, Alberton P, Aszodi A, Lundgren-Akerlund E. Integrin α10β1-selected Equine MSCs have Improved Chondrogenic Differentiation Immunomodulatory and Cartilage Adhesion Capacity. Ann. Stem Cell Res. 2019; 2:001-009.

1. An antibody or antigen-binding fragment thereof with binding specificity for integrin alpha10, wherein the antibody or antigen-binding fragment comprises:
a light chain variable region comprising
a) a CDR-L1 comprising or consisting of an amino acid sequence of SEQ ID NO: 1;
b) a CDR-L2 comprising or consisting of an amino acid sequence selected from the group consisting of SEQ ID NO: 2, and SEQ ID NO: 4; and
c) a CDR-L3 comprising or consisting of an amino acid sequence of SEQ ID NO: 5;
and/or
a heavy chain variable region comprising
d) a CDR-H1 comprising or consisting of an amino acid sequence selected from the group consisting of SEQ ID NO: 7, SEQ ID NO: 6 and SEQ ID NO: 8;
e) a CDR-H2 comprising or consisting of an amino acid sequence of SEQ ID NO: 9; and
f) a CDR-H3 comprising or consisting of an amino acid sequence of SEQ ID NO: 10.

2. The antibody or antigen-binding fragment thereof according to claim 1, wherein the antibody or antigen-binding fragment comprises:
a light chain variable region comprising
a) a CDR-L1 consisting of an amino acid sequence of SEQ ID NO: 1;
b) a CDR-L2 consisting of an amino acid sequence selected from the group consisting of SEQ ID NO: 2 and SEQ ID NO: 4; and
c) a CDR-L3 consisting of an amino acid of SEQ ID NO: 5;
and/or
a heavy chain variable region comprising
a) a CDR-H1 consisting of an amino acid sequence selected from the group consisting of SEQ ID NO: 7, SEQ ID NO: 6 and SEQ ID NO: 8;
b) a CDR-H2 consisting of an amino acid sequence of SEQ ID NO: 9; and
c) a CDR-H3 consisting of an amino acid sequence of SEQ ID NO: 10.

3. The antibody or antigen-binding fragment thereof according to any one of the preceding claims wherein the integrin alpha10beta1 is human integrin alpha10beta1.

4. The antibody or antigen-binding fragment thereof according to any one of the preceding claims, wherein the integrin alpha10 polypeptide is a part of an integrin alpha10 beta1 heterodimer.

5. The antibody or antigen-binding fragment thereof according to any one of the preceding claims wherein the integrin alpha10beta1 is expressed on the surface of a cell.

6. The antibody or antigen-binding fragment thereof according to any one of the preceding claims wherein the antibody or antigen-binding fragment thereof binds to the extracellular I-domain of integrin alpha10beta1.

7. The antibody or antigen-binding fragment according to any one of the preceding claims, wherein the antibody or antigen-binding fragment thereof comprises a light chain variable region comprising or consisting of
a) the amino acid sequence of SEQ ID NO: 13,
or an amino acid sequence having at least 85% sequence identity to SEQ ID NO: 13, for example at least 90%, 95%, 98% or 99% sequence identity; or
b) an amino acid sequence selected from the group consisting of SEQ ID NO: 11, SEQ ID NO:12; SEQ ID NO: 13; SEQ ID NO: 14 and SEQ ID NO:15, or an amino acid sequence having at least 85% sequence identity to SEQ ID NO: 11, SEQ ID NO:12; SEQ ID NO: 13; SEQ ID NO: 14 and SEQ ID NO:15, for example at least 90%, 95%, 98% or 99% sequence identity to any one of SEQ ID NO: 11, SEQ ID NO:12; SEQ ID NO: 14 and SEQ ID NO:15.

8. The antibody or antigen-binding fragment according to any one of the preceding claims, wherein the antibody or antigen-binding fragment thereof comprises a light chain variable region comprising or consisting of the amino acid sequence of SEQ ID NO: 13, or an amino acid sequence having at least 85% sequence identity to SEQ ID NO: 13, for example at least 90%, 95%, 98% or 99% sequence identity.

9. The antibody or antigen-binding fragment thereof according to any one of the preceding claims, wherein the antibody or antigen-binding fragment thereof comprises a heavy chain variable region comprising or consisting of
a) the amino acid sequence of SEQ ID NO: 19; or an amino acid sequence having at least 85% sequence identity to SEQ ID NO: 19 for example at least 90%, 95%, 98% or 99% sequence identity; or
b) an amino acid sequence selected from the group consisting of SEQ ID NO: 16, SEQ ID NO: 17, SEQ ID NO: 18, and SEQ ID NO: 20; or an amino acid sequence having at least 85% sequence identity to SEQ ID NO: 16, SEQ ID NO: 17, SEQ ID NO: 18, and SEQ ID NO: 20, for example at least 90%, 95%, 98% or 99% sequence identity to any one of SEQ ID NO: 16, SEQ ID NO: 17, SEQ ID NO: 18, and SEQ ID NO: 20.

10. The antibody or antigen-binding fragment thereof according to any one of the preceding claims, wherein the antibody or antigen-binding fragment thereof comprises a heavy chain variable region comprising or consisting of the amino acid sequence of SEQ ID NO: 19; or an amino acid sequence having at least 85% sequence identity to SEQ ID NO: 19 for example at least 90%, 95%, 98% or 99% sequence identity.

11. The antibody or antigen-binding fragment thereof according to any one of the preceding claims, wherein the antibody or antigen-binding fragment thereof comprises a light chain variable region comprising
a) a CDR-L1 comprising or consisting of an amino acid sequence of SEQ ID NO: 1;
b) a CDR-L2 comprising or consisting of an amino acid sequence selected from the group consisting of SEQ ID NO: 2, and
c) a CDR-L3 comprising or consisting of an amino acid sequence of SEQ ID NO: 5;
and
a heavy chain variable region comprising
a) a CDR-H1 comprising or consisting of an amino acid sequence selected from the group consisting of SEQ ID NO: 7,
b) a CDR-H2 comprising or consisting of an amino acid sequence of SEQ ID NO: 9; and
c) a CDR-H3 comprising or consisting of an amino acid sequence of SEQ ID NO: 10;
or an amino acid sequence having at least 70% sequence identity to SEQ ID NO: 1, SEQ ID NO: 2, SEQ ID NO: 5, SEQ ID NO: 7, or SEQ ID NO: 9, SEQ ID NO: 10, for example at least 80%, 85%, 90%, 95%, 98% or 99% sequence identity.

12. The antibody or antigen-binding fragment thereof according to any one of the preceding claims, wherein the antibody or antigen-binding fragment thereof comprises a light chain variable region which comprises or consists of SEQ ID NO: 13 and a heavy chain variable region which comprises or consists of SEQ ID NO: 19.

13. The antibody or antigen-binding fragment thereof according to any one of the preceding claims, wherein the antibody or antigen-binding fragment thereof is capable of inhibiting cell adhesion, cell proliferation, cell growth, cell migration, cell survival, or any combination thereof.

14. The antibody or antigen-binding fragment thereof according to any one of the preceding claims, wherein the antibody or antigen-binding fragment thereof is capable of inhibiting metastasis.

15. The antibody or antigen-binding fragment thereof according to any one of the preceding claims, wherein any one of the amino acids of the framework region of the light chain variable region has been altered for another amino acid, with the proviso that no more than 5 amino acids have been so altered, such as 4 amino acids, no more than 3 amino acids, such as 2 amino acids or no more than 1 amino acid.

16. The antibody or antigen-binding fragment thereof according to any one of the preceding claims, wherein any one of the amino acids of the framework region of the heavy chain variable region has been altered for another amino acid, with the proviso that no more than 5 amino acids have been so altered, such as 4 amino acids, no more than 3 amino acids, such as 2 amino acids or no more than 1 amino acid.

17. The antibody or antigen-binding fragment thereof according to any one of the preceding claims, wherein any one of the amino acids of the framework region of the light chain variable region and/or the heavy chain variable region has been altered for another amino acid, with the proviso that no more than 5 amino acids have been so altered, such as 4 amino acids, no more than 3 amino acids, such as 2 amino acids or no more than 1 amino acid.

18. The antibody or antigen-binding fragment thereof according to any one of the preceding claims, wherein the antibody or antigen-binding fragment thereof comprises a light chain constant region, or part thereof.

19. The antibody or antigen-binding fragment thereof according to claim 18, wherein the light chain constant region is of a kappa or lambda light chain.

20. The antibody or antigen-binding fragment thereof according to any one of claims 18 to 19, wherein the light chain constant region is of a kappa light chain.

21. The antibody or antigen-binding fragment thereof according to any one of the preceding claims, wherein the antibody or antigen-binding fragment thereof comprises a heavy chain constant region, or part thereof.

22. The antibody or antigen-binding fragment thereof according to claim 21, wherein the heavy chain constant region is of a human immunoglobulin subclass selected from the group consisting of IgG1, IgG2, IgG3 and IgG4.

23. The antibody or antigen-binding fragment thereof according to any one of claims 21 to 22, wherein the heavy chain constant region is of human immunoglobulin subclass IgG1.

24. The antibody or antigen-binding fragment thereof according to any one of the preceding claims, wherein any one of the amino acids of the light chain constant region and/or the heavy chain constant region has been altered for another amino acid, with the proviso that no more than 5 amino acids have been so altered, such as 4 amino acids, no more than 3 amino acids, such as 2 amino acids or no more than 1 amino acid.

25. The antibody or antigen-binding fragment thereof according to any one of the preceding claims wherein the antibody or antigen-binding fragment thereof comprises or consists of
a light chain variable region according to any one of claims 18 to 20, or 24; and/or
a heavy chain variable region according to any one of claims 21 to 24;

26. The antibody or antigen-binding fragment thereof according to any one of the preceding claims, wherein the antibody or antigen-binding fragment thereof comprises an Fc region.

27. The antibody or antigen-binding fragment thereof according to any one of the preceding claims comprising or consisting of an intact antibody.

28. The antibody or antigen-binding fragment thereof according to any one of the preceding claims comprising or consisting of an antigen-binding fragment selected from the group consisting of Fv fragments (e.g. single chain Fv and disulphide-bonded Fv), Fab-like fragments (e.g. Fab fragments, Fab′ fragments and F(ab)₂ fragments) and domain antibodies (e.g. single V_{H} variable domains or V_{L} variable domains).

29. The antibody or antigen-binding fragment thereof according to any one of the preceding claims, wherein the antibody or antigen-binding fragment thereof is capable of inhibiting signalling of integrin alpha10beta1.

30. The antibody or antigen-binding fragment thereof with binding specificity for integrin alpha10beta1 is capable of inhibiting cell adhesion, cell proliferation, cell growth, cell migration, cell survival, or any combination thereof, of cells expressing integrin alpha10beta1.

31. The antibody or antigen-binding fragment thereof according to any one of claims 29 to 30, wherein inhibiting is essentially complete inhibition.

32. The antibody or antigen-binding fragment thereof according to any one of claims 29 to 30, wherein inhibiting is partial inhibition.

33. The antibody or antigen-binding fragment thereof for use according to any one of the preceding claims, wherein the antibody or antigen-binding fragment thereof is conjugated to an additional moiety.

34. The antibody or antigen-binding fragment thereof for use according to any one of the preceding claims, wherein the additional moiety comprises a detectable moiety, such as a detectable moiety selected from the group consisting of a fluorophore, an enzyme and a radioactive tracer or radioisotope.

35. The antibody or antigen-binding fragment thereof for use according to any one of the preceding claims, wherein the additional moiety comprises a cytotoxic moiety.

36. The antibody or antigen-binding fragment thereof for use according to any one of the preceding claims, wherein the cytotoxic moiety is selected from a group consisting of a toxin, a chemotherapeutic agent and a radioactive agent, or combinations thereof.

37. The antibody or antigen-binding fragment thereof for use according to any one of the preceding claims, wherein the cytotoxic moiety is a toxin.

38. The antibody or antigen-binding fragment thereof for use according to any one of the preceding claims wherein said toxin is selected from the group selected from microtubule toxins, DNA toxins and transcription toxins.

39. The antibody or antigen-binding fragment thereof for use according to any one of the preceding claims wherein said microtubule toxins are selected from the group consisting of Auristatin-based toxins, Maytansinoid-based toxins, Tubulysins-based toxins and Eribulin.

40. The antibody or antigen-binding fragment thereof for use according to any one of the preceding claims wherein said transcription toxin is an RNA polymerase II inhibiting agent.

41. The antibody or antigen-binding fragment thereof for use according to any one of the preceding claims wherein said transcription toxin is selected from the group consisting of Doxorubicin, Doxorubicin derivatives and Amanitin.

42. The antibody or antigen-binding fragment thereof for use according to any one of the preceding claims wherein said Doxorubicin derivative is 3′-deamino-3″-4′-anhydro-[2″(S)-methoxy-3″(R)-hydroxy-4″-morpholinyl]doxorubicin.

43. The antibody or antigen-binding fragment thereof for use according to any one of the preceding claims, wherein the transcription toxin is selected from the group consisting of shiga and shiga-like toxins; type I ribosome inactivating proteins, type II ribosome inactivating proteins and saporin, or combinations thereof.

44. The antibody or antigen-binding fragment thereof for use according to any one of the preceding claims, wherein the type I ribosome inactivating protein is trichosanthin and/or luffin.

45. The antibody or antigen-binding fragment thereof for use according to any one of the preceding claims, wherein the type II ribosome inactivating protein is ricin, agglutinin and/or abrin.

46. The antibody or antigen-binding fragment thereof for use according to any one of the preceding claims, wherein the transcription toxin is selected from the group consisting of shiga and shiga-like toxins; type I ribosome inactivating proteins, type II ribosome inactivating proteins and saporin, or combinations thereof.

47. The antibody or antigen-binding fragment thereof for use according to any of the preceding claims, wherein the cells are malignant cells and/or tumor-associated cells.

48. The antibody or antigen-binding fragment thereof for use according to any of the preceding claims, wherein the malignant cells or tumor-associated cells are cancer associated fibroblast (CAFs), stromal cells, stem cells and/or stem-like cells and/or cells of the tumor microenvironment such as tumor-associated macrophages (TAMs), immune cells, endothelial cells.

49. The antibody or antigen-binding fragment thereof for use according to any of the preceding claims, wherein the treatment is initiated upon detection of an integrin alpha10 polypeptide in a cancer cell in a tumor of a subject.

50. The antibody or antigen-binding fragment thereof for use according to any of the preceding claims, wherein the antibody or antigen-binding fragment is administered to an individual in need thereof in combination with radiation therapy and/or surgical removal of cancer.

51. The antibody or antigen-binding fragment thereof for use according to any of the preceding claims, wherein the antibody or antigen-binding fragment is administered to an individual in need thereof prior to radiation therapy and/or surgical removal of cancer.

52. The antibody or antigen-binding fragment thereof for use according to any of the preceding claims, wherein the antibody or antigen-binding fragment is administered to an individual in need thereof after radiation therapy and/or surgical removal of cancer.

53. A polynucleotide encoding an antibody or antigen-binding fragment thereof according to any one of the preceding claims or a component polypeptide chain thereof.

54. The polynucleotide according to claim 53, wherein the molecule is a cDNA molecule.

55. The polynucleotide according to any one of claims 53 to 54, encoding an antibody light chain or variable region thereof.

56. The polynucleotide according to any one of claims 53 to 54 encoding an antibody heavy chain or variable region thereof.

57. The polynucleotide according to any one of claims 53 to 54 encoding an antibody according to claim 11.

58. A vector comprising a polynucleotide according to any one of the claims to claims 53-57.

59. The vector according to claim 58 wherein the vector is an expression vector.

60. A recombinant host cell comprising a polynucleotide according to any one of claims 53 to 57 or a vector according to any one of claims 58 to 59.

61. The host cell according to claim 60, wherein the host cell is a bacterial cell.

62. The host cell according to claim 60, wherein the host cell is a yeast cell.

63. The host cell according to claim 60, wherein the host cell is a mammalian cell.

64. The host cell according to claim 60, wherein the host cell is a human cell.

65. A method for producing an antibody or antigen-binding fragment thereof according to any one of claims 1 to 52, the method comprising culturing a host cell according to any one of the claims 60 to 64 comprising the polynucleotide according to any one of the claims 53 to 57 or the vector according to claim 58 to 59, under conditions which permit expression of the encoded antibody or antigen-binding fragment thereof.

66. A pharmaceutical composition comprising
the antibody or antigen-binding fragment thereof according to any one of claims 1 to 52,
the polynucleotide according to any one of claims 53 to 57,
the vector according to any one of claims 58 to 59, and/or
the recombinant host cell according to any one of claims 60 to 64,
in a pharmaceutical composition, wherein the composition further comprises a pharmaceutically-acceptable buffer, diluent, carrier or excipient.

67. The composition according to claim 66, wherein the composition comprises an effective amount of the antibody or antigen-binding fragment thereof, the polynucleotide, the vector, and/or the recombinant host cell.

68. The composition according to any one of claims 66 to 67, for use in the diagnosis and/or treatment of a cancer form selected from the group consisting of breast cancer, brain cancer, cancer of the Central Nervous System (CNS), lung cancer, prostate cancer, pancreatic cancer, skin cancer, lymphoma and sarcoma, or a metastasis of any one of said cancer forms.

69. The composition according to any one of claims 66 to 68 adapted for parenteral delivery.

70. The composition according to claim 69, wherein the parenteral delivery is intravenous delivery, topical delivery, subcutaneous delivery and/or intramuscular delivery.

71. An antibody or antigen-binding fragment thereof according to any one of claims 1 to 52, a polynucleotide according to any one of the claims 53 to 57, a vector according to any one of the claims 58 to 59, a recombinant host cell according to any one of claims 60 to 64, and/or a composition according to any one of claims 66 to 70, for use in medicine.

72. An antibody or antigen-binding fragment thereof according to any one of claims 1 to 52, a polynucleotide according to any one of the claims 53 to 57, a vector according to claim to any one of the claims 58 to 59, a recombinant host cell according to any one of claims 60 to 64, and/or a composition according to any one of claims 66 to 70,
for use in the prevention, treatment, alleviation, detection and/or diagnosis of a disease or disorder susceptible to treatment with an inhibitor of integrin alpha10, and/or
wherein the disease or disorder is associated with cells expressing integrin alpha10beta1.

73. The antibody or antigen-binding fragment thereof, the polynucleotide, the vector and/or the composition for use according to claim 72, wherein the disease or disorder is a neoplastic disease or disorder.

74. The antibody, the antigen-binding fragment thereof, the polynucleotide, the vector, the host cell or the composition for use according to any one of claims 72 to 73,
wherein the neoplastic disease or disorder is solid tumor selected from the group consisting of prostate cancer, breast cancer, brain cancer, cancer of the CNS, lung cancer, melanomas, pancreatic cancer, skin cancer, lymphoma, sarcoma, or a metastasis of any one of said cancer forms.

75. The antibody, the antigen-binding fragment thereof, the polynucleotide, the vector, the host cell or the composition for use according to any one of claims 72 to 74, wherein the breast cancer is selected from the group consisting of triple negative breast cancer and inflammatory breast cancer.

76. The antibody, the antigen-binding fragment thereof, the polynucleotide, the vector, the host cell or the composition for use according to claim 75, wherein the triple negative breast cancer is selected from the group consisting of basal-like 1 breast cancer, basal-like 2 breast cancer, claudin-low breast cancer, metaplastic breast cancer (MBC), interferon-rich breast cancer, immunomodulatory breast cancer, mesenchymal breast cancer, mesenchymal stem-like breast cancer, luminal androgen receptor breast cancer and unstable breast cancer.

77. The antibody, the antigen-binding fragment thereof, the polynucleotide, the vector, the host cell or the composition for use according to any one of claims 72 to 74, wherein the lung cancer is selected from the group consisting of squamous cell lung carcinoma, lung adenocarcinoma, large cell lung carcinoma and small-cell lung carcinoma.

78. The antibody, the antigen-binding fragment thereof, the polynucleotide, the vector, the host cell or the composition for use according to any one of claims 72 to 74, wherein the prostate cancer is small cell neuroendocrine carcinoma (SCNC) or castrate-resistant prostate cancer (CRPC).

79. The antibody, the antigen-binding fragment thereof, the polynucleotide, the vector, the host cell or the composition for use according to any one of claims 72 to 74, wherein the pancreatic cancer is an exocrine tumor or an endocrine tumor, such as wherein the pancreas cancer is an exocrine tumor selected from the group consisting of ductal adenocarcinoma, acinar cell carcinoma, adeno-squamous carcinoma, intraductal papillary mucinous neoplasm (IPMN) and Pancreatic intraepithelial neoplasia.

80. The antibody, the antigen-binding fragment thereof, the polynucleotide, the vector, the host cell or the composition for use according to claim 79, wherein the pancreas cancer is an endocrine tumor selected from the group consisting of neuroendocrine tumor, gastrinoma, glucagonoma, insulinoma, somatostatinoma, VIPoma, and non-functional Islet cell tumor, such as wherein the neuroendocrine tumor is a grade I, grade II or grade III pancreatic cancer.

81. The antibody, the antigen-binding fragment thereof, the polynucleotide, the vector, the host cell or the composition for use according to any one of claims 72 to 74, wherein the brain cancer and/or the cancer of the CNS is selected from the group consisting of:
a) Tumours of neuroepithelial tissue selected from
ii) Astrocytic tumours selected from
Pilocytic astrocytoma, Pilomyxoid astrocytoma, Subependymal giant cell astrocytoma, Pleomorphic xanthoastrocytoma, Diffuse astrocytoma, Anaplastic astrocytoma, Glioblastoma, Giant cell glioblastoma, Gliosarcoma, Gliomatosis cerebri, and
ii) Oligodendroglial tumours selected from Oligodendroglioma and Anaplastic oligodendroglioma, and
iii) Oligoastrocytic tumours selected from Oligoastrocytoma and Anaplastic oligoastrocytoma, and
iv) Ependymal tumours selected from Subependymoma, Myxopapillary ependymoma, Ependymoma, Anaplastic ependymoma, and
v) Choroid plexus tumours selected from Choroid plexus papilloma, Atypical choroid plexus papilloma, and Choroid plexus carcinoma, and
vi) Other neuroepithelial tumours selected from Astroblastoma, Choroid glioma of the third ventricle, and Angiocentric glioma and,
vii) Neuronal and mixed neuronal-glial tumours selected from Dysplastic gangliocytoma of cerebellum (Lhermitte-Duclos), Desmoplastic infantile astrocytoma/ganglioglioma, Dysembryoplastic neuroepithelial tumour, Gangliocytoma, Ganglioglioma, Anaplastic ganglioglioma, Central neurocytoma, Extraventricular neurocytoma, Cerebellar liponeurocytoma,
Papillary glioneuronal tumour, Rosette-forming glioneuronal tumour of the fourth ventricle, and Paraganglioma, and
viii) Tumours of the pineal region selected from Pineocytoma, Pineal parenchymal tumour of intermediate differentiation, Pineoblastoma, and Papillary tumours of the pineal region, and
ix) Embryonal tumours selected from Medulloblastoma, Medulloblastoma with extensive nodularity, Anaplastic medulloblastoma, CNS Primitive neuroectodermal tumour, CNS Neuroblastoma, and Atypical teratoid/rhabdoid tumour), and
b) Tumours of cranial and paraspinal nerves selected from
i) Schwannoma, ii) Neurofibroma, iii) Perineurioma, and iv) Malignant peripheral nerve sheath tumour (MPNST), and
c) Tumours of the meninges selected from
i) Tumours of meningothelial cells, selected from Meningioma, Atypical meningioma, Anaplastic meningioma,
ii) Mesenchymal tumours selected from Lipoma, Angiolipoma, Hibernoma, Liposarcoma, Solitary fibrous tumour, Fibrosarcoma, Malignant fibrous histiocytoma, Leiomyoma, Leiomyosarcoma, Rhabdomyoma, Rhabdomyosarcoma, Chondroma, Chondrosarcoma, Osteoma, Osteosarcoma, Osteo-chondroma, Haemangioma, Epithelioid hemangioendothelioma, Haemangiopericytoma, Anaplastic haemangiopericytoma, and Angiosarcoma, Kaposi Sarcoma, Ewing Sarcoma—PNET,
iii) Primary melanocytic lesions selected from
Diffuse melanocytosis, Melanocytoma, Malignant melanoma, Meningeal melanomatosis, and
iv) Other neoplasms related to the meninges such as Haem-angioblastoma, and
d) Tumours of the haematopoietic system selected from
i) Malignant Lymphomas, Plasmocytoma, and ii) Granulocytic sarcoma, and
e) Tumours of the sellar region selected from
ii) Craniopharyngioma, ii) Granular cell tumour, iii) Pituicytoma, and iv) Spindle cell oncocytoma of the adenohypophysis.

82. The antibody, the antigen-binding fragment thereof, the polynucleotide, the vector, the host cell or the composition for use according to any one of claim 72 to or 74, wherein the neoplastic disease or disorder is a solid tumor.

83. The antibody, the antigen-binding fragment thereof, the polynucleotide, the vector, the host cell or the composition for use according to any one of claim 72 to or 74, wherein the neoplastic disease or disorder is a lymphoma.

84. An antibody or antigen-binding fragment thereof according to any one of claims 1 to 52, a polynucleotide according to any one of the claims 53 to 57, a vector according to claim to any one of the claims 58 to 59, a recombinant host cell according to any one of claims 60 to 64, and/or a composition according to any one of claims 66 to 70,
for use in inhibiting cell migration, cell proliferation, cell growth, cell survival and/or cell adhesion of cells expressing integrin alpha10beta1.

85. An in vitro method for the detection of cells expressing integrin alpha10beta1 in a subject, the method comprising:
(a) providing a sample of cells from a subject to be tested, such as biopsy tissue or blood sample;
(b) optionally, extracting and/or purifying the cells present in the sample;
(c) contacting an antibody or antigen-binding fragment thereof according to any one of claims 1 to 52 with cells present in the sample;
(d) determining whether the antibody or antigen-binding fragment thereof binds to the cells
wherein the binding of the antibody or antigen-binding fragment thereof to the cells is indicative of the presence of a disease or disorder associated with cells expression integrin alpha10 in the tissue of a subject.

86. An in vitro method for identifying a patient with a disease or disorder associated with cells expressing integrin alpha10 who would benefit from treatment with an antibody or antigen-binding fragment thereof according to any one of claims 1 to 52, the method comprising:
(a) providing a sample of cells, such as biopsy tissue or blood sample from a patient to be tested;
(b) optionally, extracting and/or purifying the cells present in the sample;
(c) contacting an antibody or antigen-binding fragment thereof according to any one of claims 1 to 52 with cells present in the sample;
(d) determining whether the antibody or antigen-binding fragment thereof binds to the cells
wherein the binding of the antibody or antigen-binding fragment thereof to cells expressing integrin alpha 10 is indicative of a patient who would benefit from treatment with an antibody or antigen-binding fragment thereof according to any one of claims 1 to 52.

87. A method for treating a patient with a disease or disorder associated with cells expressing integrin alpha10, the method comprising:
a) selecting a patient identified as having a disease or disorder associated with cells expressing integrin alpha10 according to any one of claims 85 to 86;
b) administering to said patient a therapeutic agent effective in the treatment of said disease or disorder.

88. A method for the detection of cells expressing integrin alpha10, the method comprising:
a) contacting an antibody or antigen-binding fragment thereof according to any one of claims 1 to 52 with cells to be analysed for their expression of integrin alpha 10;
b) determining whether the antibody or antigen-binding fragment thereof binds to the cells
wherein the binding of the antibody or antigen-binding fragment thereof to the cells is indicative of the presence of a disease or disorder associated with cells expression integrin alpha 10 in the tissue of a subject.

89. The method according to claim 88, wherein the method is an in vivo method or an in vitro method.

90. A method for in vivo imaging the expression of the integrin alpha10beta1 in a mammal, the method comprising the steps of
a) Providing a mammal,
b) Providing an antibody or antigen-binding fragment thereof according to any one of claims 1 to 52,
c) administering the antibody or antigen-binding fragment thereof according to any one of claims 1 to 52 to the mammal so as to allow the antibody or a fragment thereof to bind to an extracellular domain of integrin alpha10beta1 of cells in said mammal,
d) optionally adding a second labelled antibody or a fragment thereof to the sample, wherein the second antibody or a fragment thereof binds to the antibody or a fragment thereof in c),
e) detecting the antibody or antigen-binding fragment thereof according to any one of claims 1 to 52 of said cells in c), or optionally detecting the second labelled antibody or a fragment thereof in d) bound to the antibody or a fragment thereof, and
f) creating an image of the detected antibody or a fragment thereof, thereby imaging the expression of integrin alpha10beta1 on cells in a mammal in vivo.

91. A method for in vitro detection of integrin alpha10 or a fragment thereof in a sample obtained from a mammal, the method comprising the steps of
a) Providing a sample obtained from a mammal,
b) Providing an antibody or antigen-binding fragment thereof according to any one of claims 1 to 52,
c) Contacting the antibody or antigen-binding fragment thereof with the sample so as to allow the antibody or a fragment thereof to bind to an extracellular domain of integrin alpha10, or a fragment thereof, in said sample,
d) optionally adding a second labelled antibody or a fragment thereof to the sample, wherein the second antibody or a fragment thereof binds to the antibody or a fragment thereof in c),
e) detecting the antibody or antigen-binding fragment thereof, or optionally detecting the second labelled antibody or a fragment thereof in d) bound to the antibody or a fragment thereof, and
thereby determining whether integrin alpha10 or a fragment thereof is found in the sample obtained from the mammal.
